(19) United States
(12) Patent Application Publication
(10) Pub. No.: US 2005/0250835 A1
(43) Pub. Date: Nov. 10, 2005

(54) Novel tumor-selective chemotherapeutic agents

(76) Inventor: Yuqiang Wang, Cupertino, CA (US)

Correspondence Address: FOLEY & LARDNER LLP, 111 HUNTINGTON AVENUE, 26TH FLOOR, BOSTON, MA, 02199-7610, US

(21) Appl. No.: 11/082,506

(22) Filed: Mar. 17, 2005

Related U.S. Application Data
(60) Provisional application No. 60/564,561, filed on Apr. 23, 2004.

(51) Int. Cl. A61K 31/403; C07D 45/14; C07D 43/14

(52) U.S. Cl. 514/411; 548/427

(57) ABSTRACT

The present invention relates to novel CC-1065 derivatives that bind to albumin in vitro and in vivo forming albumin-drug conjugates, and their methods of preparation and use as antitumor agents.

### RELATED APPLICATIONS

The present application claims the benefit, under 35 U.S.C. § 119, of U.S. provisional patent application Ser. No. 60/564,561, which was filed on Apr. 23, 2004, the entire contents of which are hereby incorporated herein by reference.

### FIELD OF THE INVENTION

The present invention relates to novel CC-1065 derivatives that bind to albumin in vitro and in vivo forming albumin-drug conjugates, and their methods of preparation and use as antitumor agents.

### BACKGROUND OF THE INVENTION

Cancer cells do not contain molecular targets that are completely foreign to the host. Therefore, most anticancer chemotherapies have relied primarily on the enhanced proliferative rate of cancer cells. Anticancer drugs kill the rapidly dividing tumor cells in either S or G2-M phases of the cell cycle while sparing the quiescent tumor and normal cells in G1 or G0 phases (Tannock, I. F. in DeVita et al., eds. Cancer: Principle and Practice of Oncology: 3-13, J. B. Lippincott, Philadelphia, 1989). The fraction of tumor cells that are dividing at any time varies depending upon tumor type and the growth stage of the tumor. In general, faster-cycling tumors e.g, lymphomas, testicular tumors, and some childhood tumors, are more susceptible to chemotherapy than are the more common types of solid tumors with slowly cycling or noncycling cells. However, some normal cells such as bone marrow and intestinal mucosa also cycle rapidly, making them susceptible to the toxic side effects of chemotherapeutic drugs. Thus, finding a unique property of tumor biochemistry or physiology that can be exploited to target chemotherapeutic drugs to tumors to maintain an effective concentration for longer times and thereby create a greater therapeutic advantage is important to a successful cancer therapy. There remains a need in the art for effective cancer therapeutics.

Human serum albumin (HSA) is the most well studied plasma protein. HSA is known to bind to various endogenous metabolites, metal ions, and drugs (Carter and Ho, Adv. Protein Chem. 1994, 45, 153-203; Peters, Adv. Protein Chem. 1985, 37, 161-247). Binding of drugs to serum albumin affects their metabolism, efficacy and body distribution (Herve et al., Clin. Pharmacokinet. 1994, 26, 44-58). Because HSA is biodegradable, non-toxic and non-immunogenic, it is widely used as a stabilizing component in pharmaceutical and biologic products including vaccines, recombinant therapies and coatings for medical devices. Recently, experiments demonstrate that HSA preferentially accumulates in solid tumors (Kratz and Beyer, Drug Delivery 1998, 5, 1-19). Several factors account for this preferential accumulation. Among them are (a) because of the enhanced proliferative rate, tumor cells take up albumin at a greater rate than normal cells. After lysosomal digestion, the derived amino acids from albumin serve as a source for nitrogen and energy in tumor cells; (b) the abnormal vasculature of tumors is highly permeable, which makes them taking up large molecules more efficiently than normal cells; (c) the poor lymphatic drainage of tumors cannot readily remove large molecules, leading to an accumulation of these large molecules in the tumor (Nugent and Jain, Cancer Res. 1984, 44, 38-244; Maeda, In: A. J. Domb, (ed.), Polymeric site-specific pharmacotherapy, pp. 95-116. New York: J. Wiley, 1994; Yuan et al., Cancer Res. 1995, 55, 3552-3756). This phenomenon of tumor tissues is called “enhanced permeability and retention” (EPR) (Duncan et al., Biosci. Rep. 1983, 2, 1041-1046; Matsumura and Maeda, Cancer Res. 1986, 46, 6387-6392; Fang et al., Adv. Exp. Med. Biol. 2003, 519, 29-49). A HSA prodrug can function as a reservoir of the drug for a long duration of action, resulting in an improvement of efficacy for drugs that have a relatively narrow therapeutic index (Herve et al., Clin. Pharmacokinet. 1994, 26, 44-58). Due to these unique properties, HSA is now used to target antitcancer drugs selectively to cancer to increase the drug's therapeutic index.

### SUMMARY OF THE INVENTION

The present invention provides compounds that can be used for the treatment of mammalian diseases characterized by aberrant cellular proliferation, e.g., tumors and cancers. The present invention provides a class of compounds that will form conjugates with albumin in vitro and in vivo leading to a greatly improved therapeutic efficacy, compared to the unconjugated free drugs. These compounds, having affinity for albumin, have been tested in experimental animal tumor models and have demonstrated excellent antitumor activity, with the albumin-conjugated compounds having increased antitumor activity compared to the free (unconjugated) compounds. The compounds are exemplified by a class of CC-1065 analogs that have generally the following formula (I), as well as pharmaceutically acceptable salts and formulations thereof:
CC-1065 analogue-linker-maleimide (Formula I)
wherein:
The linker is selected from the group including —C(O)R₁—, —C(O)OR₁—, —C(O)NR₂R₃—, —C(O)(CH₂)ₙ₁(OCH₂CH₂)ₙ₂— where
   ₙ₁ is 1-6, and ₙ₂ is 0-20, and —C(O)(CH₂)ₙ₃R₄(CH₂)ₙ₄— where ₙ₃ and ₙ₄ are independently 0-10;
   —C(O)(CH₂)ₙ₁R₄(OCH₂CH₂)ₙ₂— where ₙ₁ is 1-6, and ₙ₂ is 0-20,
   —C(O)(CH₂)ₙ₁R₄(OCH₂CH₂)ₙ₂R₄— where ₙ₁ is 1-6, and ₙ₂ is 0-20,
   —C(O)NR₂R₃(CH₂)ₙ₃R₄(CH₂)ₙ₄— where ₙ₃ and ₙ₄ are independently 0-10 and
   —C(O)NR₂R₃(CH₂)ₙ₅(OCH₂CH₂)ₙ₆— where ₙ₅ and ₙ₆ are independently 0-10; and
   wherein:
   R₁ is alkyl or aryl;
   R₂ and R₃ are independently H, alkyl or aryl; but R₂ and R₃ cannot simultaneously be aryl;
   R₄ is a valence bond, aryl, or alkyl containing at least one nitrogen;

The CC-1065 analogue includes a compound with the following structure (Formula II): 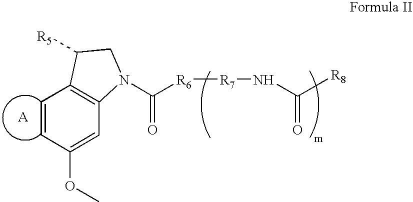
wherein:
A is a 5-6 member ring alkyl, aryl or heteroaryl;
R₅ is CH₂Cl, CH₂Br, CH₂I or CH₂OSO₂CH₃;
R₆ is a valence bond, a C₁-C₆ alkyl, a C₂-C₆ alkenyl, a C₂-C₆ alkynyl or an aryl;
R₇ and R₈ are independently selected from aryl or heteroaryl; and
M is 0-2; and
   maleimide, having generally the structure given by formula III.

Maleimide is a structure of formula III:

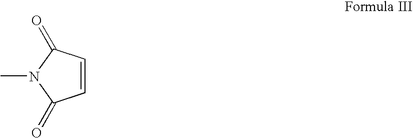

In preferred embodiments, the compounds of the present invention include those where the CC-1065 analogue of formula (I), is a compound having a structure given by one of formula IV, V or VI: 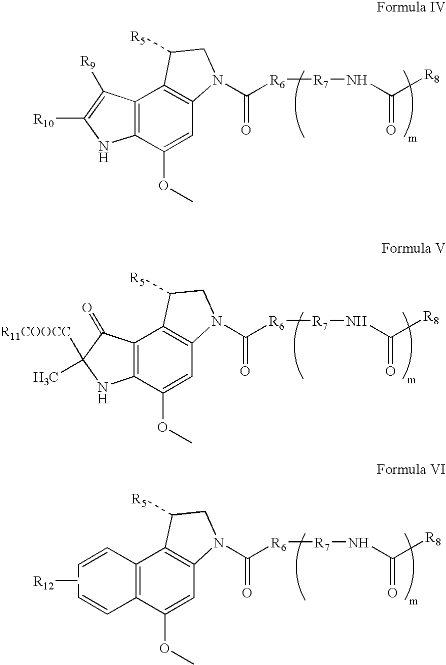
wherein:
R₅ is CH₂Cl, CH₂Br, CH₂I or CH₂OSO₂CH₃;
R₆ is a valence bond, a C₁-C₆ alkyl, a C₂-C₆ alkenyl, a C₂-C₆ alkynyl or an aryl;
   R₇ and R₈ are independently selected from aryl or heteroaryl;
M is 0-2;
R₉ is H, C₂-C₆ alkyl, C(O)-alkyl, C(O)O-alkyl;
R₁₀ is H, C₂-C₆ alkyl;
R₁₁ is CH₃ or CF₃;
R₁₂ is H, NH₂, NO₂, O-alkyl, NH-alkyl, N(alkyl)₂, NHC(O)-alkyl, ONO₂, F, Cl, Br, I, OH, OCF₃, OSO₂CH₃, CO₂H, CO₂-alkyl, CO₂CF₃ or CN.

In certain embodiments, the linker is selected from a group which includes: —C(O)R₁—, —C(O)OR₁—, —C(O)NR₂R₃—, —C(O)(CH₂)ₙ₁(OCH₂CH₂)ₙ₂— and ₙ₁ is 1-6, and ₙ₂ is 0-20,
—C(O)(CH₂)ₙ₃R₄(CH₂)ₙ₄—, where ₙ₃ and ₙ₄ are independently 0-10,
—C(O)(CH₂)ₙ₁R₄(OCH₂CH₂)ₙ₂—, where ₙ₁ is 1-6, and ₙ₂ is 0-20,
—C(O)(CH₂)ₙ₁(OCH₂CH₂)ₙ₂R₄—, where ₙ₁ is 1-6, and ₙ₂ is 0-20,
—C(O)NR₂R₃(CH₂)ₙ₃R₄(CH₂)ₙ₄—, where ₙ₃ and ₙ₄ are independently 0-10 or
—C(O)NR₂R₃(CH₂)ₙ₅(OCH₂CH₂)ₙ₆—, where ₙ₅ and ₙ₆ are independently 0-10;

In another preferred embodiment the compounds of the present invention include those where the CC-1065 analogue is a compound having a structure given by formula VI: 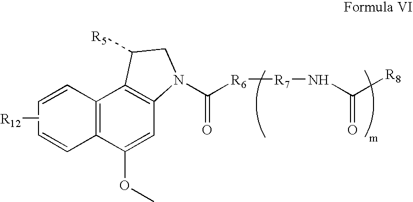
wherein:
the linker is —C(O)R₁— and R₁ is alkyl;
R₅ is CH₂Cl, CH₂Br, CH₂I or CH₂OSO₂CH₃;
R₆ is a valence bond or CH═CH;
R₇ and R₈ are independently heteroaryl;
R₁₂ is H;

A preferred antitumor composition having affinity for albumin according to the present invention is exemplified by the structure given as (+)-YW-391. shown below:

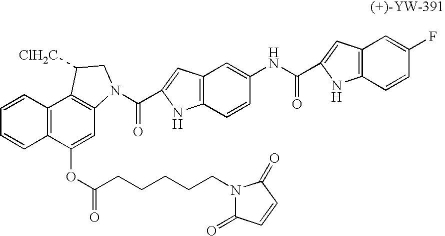

Another preferred antitumor composition having affinity for albumin according to the present invention is exemplified by the structure given as (+)-YW-392. shown below:

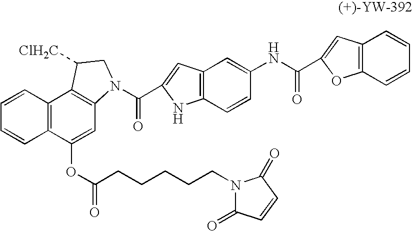

The antiproliferative compounds described herein have affinity for albumin, and provide therapeutic and prophylactic compounds that are efficacious in the treatment of mammalian diseases characterized by aberrant cellular proliferation, for example, diseases such as tumors, polyps, endometriosis, leukemias, autoimmune diseases, cancers and the like. Specific diseases that are amenable to treatment with the compounds of the present invention include those illustrated in the examples, e.g., ovarian cancer, lung cancer, and leukemia. Treatment methods include administering the compound(s) to a patient in a pharmaceutically acceptable preparation, in a dose that is effective in inhibiting such aberrant cellular proliferation. Accordingly, the invention provides a method for treating leukemia, ovarian cancer, or lung cancer in a subject comprising administering to a subject having cancer, from 1 microgram/kg to 100 micrograms/kg, and more preferably from 1 microgram/kg to about 500 micrograms/kg of the antiproliferative compounds, wherein further proliferation of the cancer is inhibited, and preferably the tumor displays a partial response or complete response to the treatment.

The invention includes synthetic processes for making the antiproliferative compounds. Also included within the scope of the invention are pharmaceutical formulations of the compounds. For example, pharmaceutically acceptable salts of the compounds, e.g., (+)-YW-391, (+)-YW-392 and the like, can be prepared for administration to human subjects. Likewise, the compounds can be preconjugated to albumin. Similarly, pharmaceutically acceptable formulations can be prepared, which incorporate the compounds, e.g., (+)-YW-391 and/or (+)-YW-392 along with appropriate excipients. The compounds of the present invention may be administered independently or in combination, and additionally may be given with other pharmaceutical actives.

The compounds of the present invention can also be used as an adjuvant to conventional cancer therapy to treat apoptosis-resistant tumors and in the treatment of other diseases to overcome drug resistance. The compounds of the present invention can be administered simultaneously or sequentially with at least one conventional cancer therapy. The conventional cancer therapy can be radiation therapy, chemotherapy, and/or biologic therapy. Preferred chemotherapy includes an antimetabolite, an alkylating agent, a plant alkaloid, and an antibiotic. Preferred antimetabolite includes methotrexate, 5-fluorouracil, 6-mercaptopurine, cytosine arabinoside, hydroxyurea, and 20-chlorodeoxyadenosine. Preferred alkylating agents includes cyclophosphamide, melphalan, busulfan, cisplatin, carboplatin, chlorambucil, and nitrogen mustards. Preferred plant alkaloid includes vincristine, vinblastine, and VP-16. Preferred antibiotic includes doxorubicin, daunorubicin, mitomycin c, and bleomycin. Alternate preferred chemotherapy includes decarbazine, mAMSA, hexamethylmelamine, mitoxantrone, taxol, etoposide, dexamethasone. Preferred radiation therapy includes photodynamic therapy, radionucleotides, and radioimmunotherapy. Preferred biologic therapy includes immunotherapy, differentiating agents, and agents targeting cancer cell biology.

The above summary sets forth rather broadly certain features of the present invention in order that the detailed description thereof that follows may be understood, and in order that the present contributions to the art may be better appreciated. Other objects and features of the present invention will become apparent from the following detailed description considered in conjunction with the accompanying drawings. It is to be understood, however, that the drawings are designed solely for the purposes of illustration and not as a definition of the limits of the invention, for which reference should be made to the appended claims.

### BRIEF DESCRIPTION OF THE DRAWINGS

The present invention will be further understood from the following description with reference to the tables and figures, in which:

FIG. 1 illustrates CC-1065 and related structures;

FIG. 2 illustrates the synthesis of (+)-YW-367;

FIG. 3 shows the synthesis of (+)-YW-391;

FIG. 4 illustrates the synthesis of(+)-YW-392;

FIG. 5 shows YW-201 induces DNA fragmentation, apoptosis and cell death in leukemia cells;

FIG. 6 illustrates the anticancer activity of (+)-YW-391 in mice having JC breast cancer;

FIG. 7 shows the anticancer activity of (+)-YW-391 in mice having Lewis lung carcinoma;

FIG. 8 shows the anticancer activity of (+)-YW-391 in nude mice having SKOV-3 human ovarian cancer.

These and other objects of the present invention will be apparent from the detailed description of the invention provided below.

### DETAILED DESCRIPTION OF THE INVENTION

The following definitions are set forth to illustrate and define the meaning and scope of the various terms used to describe the invention herein.

As used herein, the term “Alkyl” refers to unsubstituted or substituted linear, branched or cyclic alkyl carbon chains of up to 15 carbon atoms. Linear alkyl groups include, for example, methyl, ethyl, n-propyl, n-butyl, n-pentyl, n-hexyl, n-heptyl and n-octyl. Branched alkyl groups include, for example, iso-propyl, sec-butyl, iso-butyl, tert-butyl and neopentyl. Cyclic alkyl (“cycloalkyl”) groups include, for example, cyclopropyl, cyclobutyl, cyclopentyl and cyclohexyl. Alkyl groups can be substituted with one or more substituents. Nonlimiting examples of such substituents include NH₂, NO₂, O-alkyl, NH-alkyl, N(alkyl)₂, NHC(O)-alkyl, ONO₂, F, Cl, Br, I, OH, OCF₃, OSO₂CH₃, CO₂H, CO₂-alkyl, CN, aryl and heteroaryl. The term “Alkyl” also refers to unsubstituted or substituted linear, branched or cyclic chains of up to 15 carbon atoms that contain at least one heteroatom (e.g., nitrogen, oxygen or sulfur) in the chain. Such linear alkyl groups include, for example, CH₂CH₂OCH₃, CH₂CH₂N(CH₃)₂ and CH₂CH₂SCH₃. Branched groups include, for example, CH₂CH(OCH₃)CH₃, CH₂CH(N(CH₃)₂)CH₃ and CH₂CH(OCH₃)CH₃. Such cyclic alkyl groups include, for example, CH(CH₂CH₂)₂O, H(CH₂CH₂)₂NCH₃, CH(CH₂CH₂)₂S, piperidino, piperidyl and piperazino. Such alkyl groups can be substituted with one or more substituents. Nonlimiting examples of such substituents include NH₂, NO₂, O-alkyl, NH-alkyl, N(alkyl)₂, NHC(O)-alkyl, ONO₂, F, Cl, Br, I, OH, OCF₃, OSO₂CH₃, CO₂H, CO₂-alkyl, CN, aryl and heteroaryl. Further the term also includes instances where a heteroatom has been oxidized, such as, for example, to form an N-oxide, ketone or sulfone.

As used herein, the term “Aryl” refers to an unsubstituted or substituted aromatic, carbocyclic group. Aryl groups are either single ring or multiple condensed ring compounds. A phenyl group, for example, is a single ring, aryl group. A naphthyl group exemplifies an aryl group with multiple condensed rings. Aryl groups can be substituted with one or more substituents. Nonlimiting examples of such substituents include NH₂, NO₂, O-alkyl, NH-alkyl, N(alkyl)₂, NHC(O)-alkyl, ONO₂, F, Cl, Br, I, OH, OCF₃, OSO₂CH₃, CO₂H, CO₂-alkyl, CN, aryl and heteroaryl.

As used herein, the term “heteroaryl” refers to an unsubstituted or substituted aromatic mono- or poly-cyclic group containing at least one heteroatom within a ring, e.g., nitrogen, oxygen or sulfur. For example, typical heteroaryl groups with one or more nitrogen atoms are tetrazoyl, pyrrolyl, pyridyl (e.g., 4-pyridyl, 3-pyridyl, 2-pyridyl), pyridazinyl, indolyl, quinolyl (e.g., 2-quinolyl, 3-quinolyl etc.), imidazolyl, isoquinolyl, pyrazolyl, pyrazinyl, pyrimidinyl, pyridonyl or pyridazinonyl; typical oxygen heteroaryl groups with an oxygen atom are 2-furyl, 3-furyl or benzofuranyl; typical sulfur heteroaryl groups are thienyl, and benzothienyl; typical mixed heteroatom heteroaryl groups are furazanyl, oxazolyl, isoxazolyl, thiazolyl, and phenothiazinyl. Heteroaryl groups can be substituted with one or more substituents. Nonlimiting examples of such substituents include NH₂, NO₂, O-alkyl, NH-alkyl, N(alkyl)₂, NHC(O)-alkyl, ONO₂, F, Cl, Br, I, OH, OCF₃, OSO₂CH₃, CO₂H, CO₂-alkyl, CN, aryl and heteroaryl. Further the term also includes instances where a heteroatom within the ring has been oxidized, such as, for example, to form an N-oxide, ketone or sulfone.

As used herein, the term “pharmaceutically acceptable” refers to a lack of unacceptable toxicity in a compound, such as a salt or excipient. Pharmaceutically acceptable salts include inorganic anions such as chloride, bromide, iodide, sulfate, sulfite, nitrate, nitrite, phosphate, and the like, and organic anions such as acetate, malonate, pyruvate, propionate, cinnamate, tosylate, citrate, and the like. Pharmaceutically acceptable excipients are described below, and, at length by E. W. Martin, in Remington's Pharmaceutical Sciences Mack Publishing Company (1995), Philadelphia, Pa., 19^{th} ed.

The term “mammalian cell” refers to a cell or cell line derived from a mammalian source. The term “mammalian cell proliferating disease” refers to a condition wherein mammalian cells grow and/or divide or otherwise proliferate in a way or at a rate that is aberrant, i.e., differs from that observed in a normal mammalian cell.

General Principles of Treatment

The goal of cancer treatment is first to eradicate the cancer. If this primary goal cannot be accomplished, the goal of cancer treatment shifts to palliation, the amelioration of symptoms, and preservation of quality of life while striving to extend life.

Cancer treatments are divided into four main groups: surgery, radiation therapy (including photodynamic therapy), chemotherapy (including hormonal therapy), and biologic therapy (including immunotherapy, differentiating agents, and agents targeting cancer cell biology). The modalities are often used in combination, and agents in one category can act by several mechanisms. For example, cancer chemotherapy agents can induce differentiation, and antibodies (a form of immunotherapy) can be used to deliver radiation therapy. Surgery and radiation therapy are considered local treatments, though their effects can influence the behavior of tumor at remote sites. Chemotherapy and biologic therapy are usually systemic treatments.

Cancer behaves in many ways as an organ that regulates its own growth. However, cancers have not set an appropriate limit on how much growth should be permitted. Normal organs and cancers share the property of having a population of cells in cycle and actively renewing and a population of cells not in cycle. In cancers, cells that are not dividing are heterogeneous; some have sustained too much genetic damage to replicate but have defects in their death pathways that permit their survival; some are starving for nutrients and oxygen; and some are reversibly out of cycle poised to be recruited back into cycle and expand if needed. Severely damaged and starving cells are unlikely to kill the patient. The problem is that the cells that are reversibly not in cycle are capable of replenishing tumor cells physically removed or damaged by radiation and chemotherapy.

Tumors follow a Gompertzian growth curve; the growth fraction of a neoplasm starts at 100% with the first transformed cell and declines exponentially over time until by the time of diagnosis at a tumor burden of 1 to 5×10⁹ tumor cells, the growth fraction is usually 1 to 4%. Cancers try to limit their own growth but are not completely successful at doing so. The peak growth rate occurs before the tumor is detectable. Metastases can be observed to grow more rapidly than the primary tumor, consistent with the idea that an inhibitory factor slows the growth of larger tumor masses. When a tumor recurs after surgery or chemotherapy, frequently its growth is accelerated and the growth fraction of the tumor is increased.

Principles of Chemotherapy

Candidate compounds that might have selectivity for cancer cells were suggested by the marrow-toxic effects of sulfur and nitrogen mustards and led, in the 1940s, to the first notable regressions of hematopoietic tumors following use of these compounds. As these compounds caused covalent modification of DNA, the structure of DNA was thereby identified as a potential target for drug design efforts. Biochemical studies demonstrating the requirement of growing tumor cells for precursors of nucleic acids led to studies of folate analogues. The cure of patients with advanced choriocarcinoma by methotrexate in the 1950s provided further impetus to define the value of chemotherapeutic agents in many different tumor types. This resulted in efforts to understand unique metabolic requirements for biosynthesis of nucleic acids and led to the design, rational for the time, of compounds that might selectively interfere with DNA synthesis in proliferating cancer cells. The capacity of hormonal manipulations including oophorectomy and orchiectomy to cause regressions of breast and prostate cancers, respectively, provided a rationale for efforts to interfere with various aspects of hormone function in hormone-dependent tumors. The serendipitous finding that certain poisons derived from bacteria or plants could affect normal DNA or mitotic spindle function allowed completion of the classic armamentatrium of “cancer chemotherapy agents” with proven safety and efficacy in the treatment of certain cancers.

End-Points of Drug Action

Chemotherapy agents may be used for the treatment of active and clinically apparent cancer. Tumors considered curable by conventionally available chemotherapeutic agents are listed in Table 1A. Most commonly, chemotherapeutic agents are used to address metastatic cancers. If a tumor is localized to a single site, serious consideration of surgery or primary radiation therapy should be given, as these treatment modalities may be curative as local treatments.

Chemotherapy may be employed after the failure of these modalities to eradicate a local tumor, or as part of multimodality approaches to offer primary treatment to a clinically localized tumor. In this event, it can allow organ preservation when given with radiation, as in larynx or other upper airway sites; or sensitize tumors to radiation when given, for example, to patients concurrently receiving radiation for lung or cervix cancer (Table 1B). Chemotherapy can be administered as an adjuvant to surgery (Table 1C) or radiation, a use that may have curative potential in breast, colon, or anorectal neoplasms. In this use, chemotherapy attempts to eliminate clinically unapparent tumor that may have already disseminated. Chemotherapy can be used in conventional dose regimens. In general, these doses produce reversible acute side effects primarily consisting of transient myelosuppression with or without gastrointestinal toxicity (nausea), which are readily managed. High-dose chemotherapy regimens are predicated on the observations that the concentration-effect curve for many anticancer agents is rather steep, and increased dose can produce markedly increased therapeutic effect, although at the cost of potentially life-threatening complications that require intensive support, usually in the form of bone marrow or stem cell support from the patient (autologous) or from donors matched for histocompatibility loci (allogeneic). High-dose regimens nonetheless have definite curative potential in defined clinical settings (Table 1D).

The evaluation of a chemotherapeutic agent's benefit can be assessed by carefully quantitating its effect on tumor size and using these measurements to decide objectively the basis for further treatment of a particular patient or further clinical evaluation of a drug's potential. A partial response (PR) is defined conventionally as a decrease by at least 50% in a tumor's bi-dimensional area; a complete response (CR) connotes disappearance of all tumors; progression of disease signifies increase by greater than 25% from baseline or best response; and “stable” disease fits into none of the above categories. Accordingly, the invention provides a method for treating ovarian cancer, lung cancer, or leukemia in a subject comprising administering to a subject having cancer, from 1 microgram/kg to 100 micrograms/kg, and preferably from 1 microgram/kg to 500 micrograms/kg of the compounds described herein, wherein the proliferation of the cancer is inhibited, i.e., where the tumor does not increase in mass, or where a partial or complete response is seen.

If cure is not possible, chemotherapy may be undertaken with the goal of palliating some aspect of the tumor's effect on the host. Accordingly, the invention provides a method for treating cancer in a subject comprising administering to a subject having cancer, from 1 microgram/kg to 500 micrograms/kg of the compounds described, wherein the proliferation of the cancer is inhibited and palliative effects are seen. Common tumors that have been meaningfully addressed with palliative intent are listed in Table 1E. Usually tumor-related symptoms may manifest as pain, weight loss, or some local symptom related to the tumor's effect on normal structures. Patients treated with palliative intent should be aware of their diagnosis and the limitations of the proposed treatment, have access to suitable palliative strategies in the event that no treatment is elected, and have a suitable “performance status”—according to assessment algorithms such as the one developed by Karnofsky or by the Eastern Cooperative Oncology Group (ECOG). ECOG performance status 0 (PS0) patients are without symptoms; PS1 patients have mild symptoms not requiring treatment; PS2, symptoms requiring some treatment; PS3, disabling symptoms, but allowing ambulation for greater than 50% of the day; PS4 patents have ambulation for less than 50% of the day. Only PS0 to PS2 patients are generally considered suitable for palliative (non-curative) treatment. If there is curative potential, even poor performance status patients may be treated, but their prognosis is usually inferior to those of good performance patients treated with similar regimens.

The usefulness of any drug is governed by the extent to which a given dose causes a useful result (therapeutic effect; in the case of anticancer agents, toxicity to tumor cells) as opposed to a toxic effect. The therapeutic index is the degree of separation between toxic and therapeutic doses. Really useful drugs have large therapeutic indices, and this usually occurs when the drug target is expressed in the disease-causing compartment as opposed to the normal compartment. Classically, selective toxicity of an agent for an organ is governed by the expression of an agent's target; or differential accumulation into or elimination from compartments where toxicity is experienced or ameliorated, respectively. Current antineoplastic agents have the unfortunate property that their targets are present in both normal and tumor tissues. Therefore, antineoplastic agents have relatively narrow therapeutic indices.

TABLE 1
Curability of Cancers with Chemotherapy
A. Advanced cancers with possible cure

| | |
|---|---|
| Acute lymphoid and acute myeloid leukemia | Gestational trophoblastic neoplasia |
| (pediatric/adult) | Pediatric neoplasms |
| Hodgkin's disease (pediatric/adult) | Wilm's tumor |
| Lymphomas - certain types (pediatric/adult) | Embryonal rhabdomyocarcinoma |
| Germ cell neoplasms | Ewing's sarcoma |
| Embryonal carcinoma | Peripheral neuroepithelioma |
| Teratocarcinoma | Neuroblastoma |
| Seminoma or dysgerminoma | Small cell lung carcinoma |
| Choriocarcinoma | Ovarian carcinoma |

B. Advanced cancers possibly cured by chemotherapy and radiation

| | |
|---|---|
| Squamous carcinoma (head and neck) | Carcinoma of the uterine cervix |
| Squamous carcinoma (anus) | Non-small cell lung carcinoma (stage III) |
| Breast carcinoma | Small cell lung carcinoma |

C. Cancers possibly cured with chemotherapy as adjuvant to surgery

| | |
|---|---|
| Breast carcinoma | Osteogenic sarcoma |
| Colorectal carcinoma^{a} | Soft tissue sarcoma |

D. Cancers possibly cured with “high-dose” chemotherapy with stem cell support

| | |
|---|---|
| Relapsed leukemias, lymphoid and myeloid | Chronic myeloid leukemia |
| Relapsed lymphomas, Hodgkin's and non- | Multiple myeloma |
| Hodgkin's | |

E. Cancers responsive with useful palliation, but not cure, by chemotherapy

| | |
|---|---|
| Bladder carcinoma | Cervix carcinoma |
| Chronic myeloid leukemia | Endometrial carcinoma |
| Hairy cell leukemia | Soft tissue sarcoma |
| Chronic lymphocytic leukemia | Head and neck cancer |
| Lymphoma - certain types | Adrenocortical carcinoma |
| Multiple myeloma | Islet-cell neoplasms |
| Gastric carcinoma | Breast carcinoma |

F. Tumor poorly responsive in advanced stages to chemotherapy

| | |
|---|---|
| Pancreatic carcinoma | Colorectal carcinoma |
| Biliary-tract neoplasms | Non-small cell lung carcinoma |
| Renal carcinoma | Prostate carcinoma |
| Thyroid carcinoma | Melanoma |
| Carcinoma of the vulva | Hepatocellular carcinoma |

^{a}Rectum also receives radiation therapy

In the past, agents with promise for the treatment of cancer have been detected empirically through screening for antiproliferative effects in animal or human tumors in rodent hosts or through inhibition of tumor cells growing in tissue culture. An optimal schedule for demonstrating antitumor activity in animals is defined in further preclinical studies, as is the optimal drug formulation for a given route and schedule. Safety testing in two species on an analogous schedule of administration defines the starting dose for a phase I trial in humans, where escalating doses of the drug are given until reversible toxicity is observed. Dose-limiting toxicity (DLT) defines a dose that conveys greater toxicity than would be acceptable in routine practice, allowing definition of a maximal tolerated dose (MTD). The occurrence of toxicity is correlated if possible with plasma drug concentrations. The MTD or a dose just lower than the MTD is usually the dose suitable for phase II trials, where a fixed dose is administered to a relatively homogeneous set of patients in an effort to define whether the drug causes regression of tumors. An “active” agent conventionally has partial response rates of at least 20 to 25% with reversible non-life-threatening side effects, and it may then be suitable for study in phase III trials to assess efficacy in comparison to standard or no therapy. Response is the immediate indicator of drug effect. To be clinically valuable, responses must translate into effects on overall survival or at least time to progression as important indicators of an ultimately useful drug. More recently, active efforts to quantitate effects of anticancer agents on quality of life as an important outcome are being developed. Cancer drug clinical trials conventionally use a toxicity grading scale where grade I toxicities do not require treatment; grade II often require symptomatic treatment but are not life-threatening; grade III toxicities are potentially life-threatening if untreated; grade IV toxicities are actually life-threatening; and grade V toxicities ultimately lead to patient death.

Cancer arises from genetic lesions that cause an excess of cell growth or division, with inadequate cell death. In addition, failure of cellular differentiation results in altered cellular position and capacity to proliferate while cut off from normal cell regulatory signals. Normally, cells in a differentiated state are stimulated to enter the cell cycle from a quiescent state, or “G0,” or continue after completion of a prior cell division cycle in response to environmental cues including growth factor and hormonal signals. Cells progress through G1 and enter S phase after passing through “checkpoints,” which are biochemically regulated transition points, to assure that the genome is ready for replication. One important checkpoint is mediated by the p53 tumor-suppressor gene product, acting through its up-regulation of the p21^{WAF1} inhibitor of cyclin-dependent kinase (CDK) function, acting on CDKs 4 or 6. These molecules can also be inhibited by the p16^{INK4A} and p27^{KIP1} CDK inhibitors and, in turn, are activated by cyclins of the D family (which appear during G1) and the proper sequence of regulatory phosphorylations. Activated CDKs 4 or 6 phosphorylate and thus inactivate the product of the retinoblastoma susceptibility gene, pRb, which in its nonphosphorylated state complexes with transcription factors of the E2F family. Phosphorylated pRb releases E2Fs, which activate genes important in completing DNA replication during S phase, progression through which is promoted by CDK2 acting in concert with cylins A and E. During G2, another checkpoint occurs, in which the cell assures the completion of correct DNA synthesis. Cells then progress into M phase under the influence of CDK1 and cyclin B. Cells may then go on to a subsequent division cycle or enter into a quiescent, differentiated state.

Biologic Basis for Cancer Chemotherapy

The classic view of how cancer chemotherapeutic agents cause regressions of tumors focused on models such as the L1210 murine leukemia system, where cancer cells grow exponentially after inoculation into the peritoneal cavity of an isogenic mouse. The interaction of drug with its biochemical target in the cancer cell was proposed to result in “unbalanced growth” that was not sustainable and therefore resulted in cell death, directly because of interacting with the drug's proximal target. Agents could be categorized as cell cycle-active, phase-specific (e.g., antimetabolites, purines, and pyrimidines in S phase; vinca alkaloids in M), and phase-nonspecific agents (e.g., alkylators, and antitumor antibiotics including the anthracyclines, actinomycin, and mitomycin), which can injure DNA at any phase of the cell cycle but appear to then block in G2 before cell division at a checkpoint in the cell cycle. Cells arrested at a checkpoint may repair DNA lesions. Checkpoints have been defined at the G1 to S transition, mediated by the tumor-suppressor gene p53 (giving rise to the characterization of p53 as a “guardian of the genome”); at the G2 to M transition, mediated by the chk1 kinase influencing the function of CDK1; and during M phase, to ensure the integrity of the mitotic spindle. The importance of the concept of checkpoints extends from the hypothesis that repair of chemotherapy-mediated damage can occur while cells are stopped at a checkpoint; therefore, manipulation of checkpoint function emerges as an important basis of affecting resistance to chemotherapeutic agents.

Resistance to drugs was postulated to arise either from cells not being in the appropriate phase of the cell cycle or from decreased uptake, increased efflux, metabolism of the drug, or alteration of the target, e.g., by mutation or overexpression. Indeed, the p170PGP (p170 P-glycoprotein; mdr gene product) was recognized from experiments with cells growing in tissue culture as mediating the efflux of chemotherapeutic agents in resistant cells. Certain neoplasms, particularly hematopoietic tumors, have an adverse prognosis if they express high levels of p170PGP, and modulation of this protein's function has been attempted by a variety of strategies.

Combinations of agents were proposed to afford the opportunity to affect many different targets or portions of the cell cycle at once, particularly if the toxic effects for the host of the different components of the combination were distinct. Combinations of agents were actually more effective in animal model systems than single agents, particularly if the tumor cell inoculum was high. This thinking led to the design of “combination chemotherapy” regimens, where drugs acting by different mechanisms (e.g., an alkylating agent plus an antimetabolite plus a mitotic spindle blocker) were combined. Particular combinations were chosen to emphasize drugs whose individual toxicities to the host were, if possible, distinct.

This view of cancer drug action is grossly oversimplified. Most tumors do not grow in an exponential pattern but rather follow Gompertzian kinetics, where the rate of tumor growth decreases as tumor mass increases. Thus, a tumor has quiescent, differentiated compartments; proliferating compartments; and both well-vascularized and necrotic regions. In addition, cell death is itself now understood to be a closely regulated process. Necrosis refers to cell death induced, for example, by physical damage with the hallmarks of cell swelling and membrane disruption. Apoptosis, or programmed cell death, refers to a highly ordered process whereby cells respond to defined stimuli by dying, and it recapitulates the necessary cell death observed during the ontogeny of the organism. Anoikis refers to death of epithelial cells after removal from the normal milieu of substrate, particularly from cell-to-cell contact. Cancer chemotherapeutic agents can cause both necrosis and apoptosis. Apoptosis is characterized by chromatin condensation (giving rise to “apoptotic bodies”); cell shrinkage; and, in living animals, phagocytosis by surrounding stromal cells without evidence of inflammation. This process is regulated either by signal transduction systems that promote a cell's demise after a certain level of insult is achieved or in response to specific cell-surface receptors that mediate cell death signals. Modulation of apoptosis by manipulation of signal transduction pathways has emerged as a basis for understanding the actions of currently used drugs and designing new strategies to improve their use.

The current view envisions that the interaction of a chemotherapeutic drug with its target causes or is itself a signal that initiates a “cascade” of signaling steps to trigger an “execution phase” where proteases, nucleases, and endogenous regulators of the cell death pathway are activated. Effective cancer chemotherapeutic agents are efficient activators of apoptosis through signal transduction pathways. While apoptotic mechanisms are important in regulating cellular proliferation and the behavior of tumor cells in vitro, in vivo it is unclear whether all of the actions of chemotherapeutic agents to cause cell death can be attributed to apoptotic mechanisms. However, as reviewed below, changes in molecules that regulate apoptosis are clearly correlated with clinical outcomes (e.g., overexpression of Bcl-2 and related proteins).

Cancer Chemotherapeutic Agents

Commonly Used Cancer Chemotherapy Agents

The commonly used cancer chemotherapy agents and the pertinent clinical aspects of their use are listed in Table 2. The drugs may be usefully grouped into three general categories: those affecting DNA, those affecting microtubules, and those acting at hormone-like receptors.

TABLE 2
Commonly Used Cancer Chemotherapy Agents

| Drug | Examples of Usual Doses |
|---|---|
| Alkylators | |
| Cyclophosphamide | 400-2000 mg/m² IV |
| | 100 mg/m² PO qd |
| Mechlorethamine | 6 mg/m² IV day 1 and day 8 |
| Chlorambucil | 1-3 mg/m² qd PO |
| Melphalan | 8 mg/m² qd × 5, PO |
| BCNU | 200 mg/m² IV |
| | 150 mg/m² PO |
| CCNU | 100-300 mg/m² PO |
| Ifosfamide | 1.2 g/m² per day qd × 5 MESNA |
| Procarbazine | 100 mg/m² per day qd × 14 |
| DTIC | 375 mg/m² IV day 1 Nausea |
| | Flulike |
| Hexamethylmelamine | 260 mg/m² per day qd × 14-21 as 4 divided oral doses |
| Cisplatin | 20 mg/m² qd × 5 IV 1 q3-4 weeks or 100-200 mg/m²/dose |
| | IV q3-4 weeks |
| Carboplatin | 365 mg/m² IV q3-4 weeks as adjusted for CrCl |
| Antitumor antibiotics | |
| Bleomycin | 15-25 mg/d qd × 5 IV bolus or continuous IV |
| Actinomycin D | 10-15 ug/kg per day qd × 5 IV bolus |
| Mithramycin | 15-20 ug/kg qd × 4-7 (hypercalcemia) |
| | or 50 ug/kg qod × 3-8 (antineoplastic) |
| Mitomycin C | 6-10 mg/m² q6 weeks |
| Etoposide (VP16-213) | 100-150 mg/m² IV qd × 3-5 d |
| | or 50 mg/m² PO qd × 21 d |
| | or up to 1500 mg/m²/of dose (high dose with stem cell |
| | support) |
| Teniposide (VM-26) | 150-200 mg/m² twice per week for 4 weeks |
| Amsacrine | 100-150 mg/m² IV qd × 5 |
| Topotecan | 20 mg/m² IV q3-4 weeks over 30 min |
| | or 1.5-3 mg/m² q3-4 weeks over 24 h |
| | or 0.5 mg/m² per day over 21 days |
| Irinotecan (CPT II) | 100-150 mg/m² IV over 90 min q3-4 weeks |
| | or 30 mg/m² per day over 120 h |
| Doxorubicin and | 45-60 mg/m² dose q3-4 weeks |
| daunorubicin | or 10-30 mg/m² dose q week |
| | or continuous-infusion regimen |
| Idarubicin | 10-15 mg/m² IV q 3 weeks |
| | or 10 mg/m² IV qd × 3 |
| Epirubicin | 150 mg/m² IV q3 weeks |
| Mitoxantrone | 12 mg/m² qd × 3 |
| | or 12-14 mg/m² q3 weeks |
| Antimetabolites | |
| Deoxycoformycin | 4 mg/m² IV every other week |
| 6-Mercaptopurine | 75 mg/m² PO |
| | or up 500 mg/m² PO (high dose) |
| 6-Thioguanine | 2-3 mg/kg per day for up to 3-4 weeks |
| Azathioprine | 1-5 mg/kg per day |
| 2-Chlorodeoxyadenosine | 0.09 mg/kg per day qd × 7 as continuous infusion |
| Hydroxyurea | 20-50 mg/kg (lean body weight) PO qd |
| | or 1-3 g/d |
| Methotrexate | 15-30 mg PO or IM qd × 3-5 |
| | or 30 mg IV days 1 and 8 |
| | or 1.5-12 g/m² per day (with leucovorin) |
| 5-Fluorouracil | 375 mg/m² IV qd × 5 |
| | or 600 mg/m² IV days 1 and 8 |
| Cytosine arabinoside | 100 mg/m² per day qd × 7 by continuous infusion |
| | or 1-3 g/m² dose IV bolus |
| Azacytidine | 750 mg/m² per week |
| | or 150-200 mg/m² per day × 5-10 (bolus) or (continuous IV) |
| Gemcitabine | 1000 mg/m² IV weekly × 7 |
| Fludarabine phosphate | 25 mg/m² IV qd × 5 |
| Asparaginase | 25,000 IU/m² q3-4 weeks |
| | or 6000 IU/m² per day qod for 3-4 weeks |
| | or 1000-2000 IU/m² for 10-20 days |
| Antimitotic agents | |
| Vincristine | 1-1.4 mg/m² per week |
| Vinblastine | 6-8 mg/m² per week |
| Vinorelbine | 15-30 mg/m² per week |
| Paclitaxel | 135-175 mg/m² per 24-h infusion |
| | or 175 mg/m² per 3-h infusion |
| | or 140 mg/m² per 96-h infusion |
| | or 250 mg/m² per 24-h infusion plus G-CSF |
| Docetaxel | 100 mg/m² per 1-h infusion q3 weeks |
| Estramustine phosphate | 14 mg/kg per day in 3-4 divided doses with water × 2 h after |
| | meals; Avoid Ca²-rich foods |

CC-1065 Class of Drugs

The majority of the currently used anticancer agents act through interference with synthesis and function of DNA, RNA, or protein, and almost all of the DNA interacting agents are major groove binders, for example, the methylating agents, chloroethylating agents, and nitrogen mustards. In contrast, DNA minor groove binders (MGBs) fit into the minor groove of the DNA double helix. MGBs have a very high degree of selectivity for thymine-adenine (TA) rich sequences, which are potential targets for anticancer agents (Marchini et al., Opin. Investig. Drugs 2002, 10, 1703-1714; Baraldi et al., Med. Res. Rev. 2004, 24, 475-528). Targeting of TA-rich sequences is more lethal than non-sequence-specific damage to DNA, requiring fewer DNA lesions per cell to inhibit cell growth (Wyatt et al., Biochemistry 1995, 34, 13034-13041; Woynarowski et al., Biochemistry 2000, 39, 9917-9927). The TA sequences appear to function as matrix attachment regions critical for cancer cell growth (Woynarowski et al., J. Biol. Chem. 2001, 276, 40555-40566). The CC-1065 class of compounds including the clinically tested adozelesin, bizelesin, carzelesin and KW-2189 are DNA minor groove binders, and are one of the most potent classes of anticancer agents ever discovered (FIG. 1). They are 100-10,000-fold more potent than doxorubicin, a widely used chemotherapeutic agents, and have a number of unique properties:

(1) They are extremely potent against tumor cells in vitro with IC₅₀ values in the picomolar range.

(2) They bind specifically to double-stranded B-DNA within the minor groove with a sequence preference for AT-rich regions and alkylate the N3 position of the 3′-adenine (Reynolds, et al., Biochemistry 1985, 24, 6228-6237). This mechanism of action differs from all clinically used antitumor drugs. They inhibit gene transcription by inhibiting binding of the TATA box binding protein to its target DNA (Chiang et al., Biochemistry 1994, 33, 7033-7040).

(3) They have a broad-spectrum of antitumor activity in vivo. For example, adozelesin is very active against L1210 leukemia, B16 melanoma, M5076 sarcoma, colon 38 carcinoma, colon CX-1 adenocarcinoma, lung LX-1 tumor, pancreas 02 carcinoma and ovarian 2780 carcinoma in mice (Li et al., Invest. New. Drugs 1991, 9, 137-148). Bizelesin is effective against P388 and L1210 leukemia, B16, UACC-62, LOX IMVI and SK-MEL-3 melanomas, CAKI-1 renal, LX-1 and Lewis lung, HT-29 colon and colon 38, pancreas 02, MCF7 and MX-1 breast carcinomas (Carter et al., Clin. Cancer Res. 1996, 2, 1143-1149). Carzelesin is effective against several colon adenocarcinomas and pediatric rhabdomyosarcomas (Houghton et al., Cancer Chemother. Pharmacol. 1995, 36, 45-52). KW-2189 is active against P388 and L1210 leukemia, B16 melanoma, colon 26 and colon 38 adenocarcinomas, LC-6 lung, ST-4 and ST-40 stomach, LI-7 liver, PAN-02 pancreas and MX-1 breast carcinomas (Kobayashi et al., Cancer Res. 1994, 54, 2404-2410).

Due to their high potency, unique mechanism(s) of action and wide spectrum of antitumor activity, several of these compounds have been tested in clinical trials. Adozelesin (Fleming et al., J. Natl. Cancer. Inst. 1994, 86, 368-372; Foster et al., Invest New Drugs 1996, 13, 321-326; Burris et al., Anticancer Drugs 1997, 8, 588-596), bizelesin (Pitot et al., Clin. Cancer Res. 2002, 8, 712-717), carzelesin (Wolff et al., Clin. Cancer Res. 1996, 2, 1717-1723; van Tellingen et al., Cancer Res. 1998, 58, 2410-2416) and KW-2189 (Alberts et al., Clin. Cancer Res. 1998, 4, 2111-2117; Small, et al., Invest. New Drugs 2000, 18, 193-197; Markovic et al., Am. J. Clin. Oncol. 2002, 25, 308-312) have completed Phase I/II clinical trials.

In clinical trials, one patient with liver cancer treated with 40 microg/m² of carzelesin for one cycle (days 1-5) had a partial remission for 8 months. This patient's lung metastasis disappeared and the primary liver cancer regressed by 50% (Wolff et al., Clin. Cancer Res. 1996, 2, 1717-1723). Unfortunately, this patient could not receive additional treatment because of myelotoxicity. Two important findings from clinical trials of carzelesin and other of CC-1065 class of drugs emerged. Firstly, no other major toxicity was found for these four drugs except myelotoxicity. This suggests that these drugs may be given at higher doses if myelotoxicity can be reduced. Secondly, carzelesin given 40 microg/m² maintained a plasma concentration of 1 ng/mL for approximately 1 h (at 5 ng/mL for 15 min, and the peak concentration was 10 ng/mL). The average IC₇₀ value against various cancer cells in vitro is 0.23 ng/mL for a 1 h exposure (Ghielmini et al., Br. J. Cancer 1997, 75, 878-883). Obviously, a concentration of 1 ng/mL of carzelesin for 1 h can kill only a small fraction of tumor cells because most tumor cells are in G₁ or G₀ phases during any 1-h period. This partially explains the ineffectiveness of carzelesin in patients.

We have synthesized and tested many derivatives of CC-1065 analogues (Wang et al., J. Med. Chem. 2000, 43, 1541; Wang et al., BMC Chemical Biology 2001, 1, 4; Wang et al., BMC Chemical Biology 2002, 2, 1; Wang et al., J. Med. Chem. 2003, 46, 634; Wang et al., Bioorg. Med. Chem. 2003, 11, 1569). These compounds have potent antitumor activity against a broad panel of tumor cells in vitro and in animal models. For example, YW-200 was highly active against all 60-cell lines used in the NCI in vitro screening program with IC₅₀ values in the 0.1-5 nM range for most cell lines (Table 3). YW-200 was also very active against the doxorubicin-resistant NCI-Dox-RES breast cancer cells with an IC₅₀ value of 8.6 nM.

TABLE 3
Cytotoxicity of YW-200 against tumor cell lines in vitro

| Panel/cell line | IC₅₀ (nM) |
|---|---|
| Leukemia | |
| CCRF-CEM | 3.14 |
| HL-60 (TB) | 1.40 |
| K-562 | 3.76 |
| MOLT-4 | 0.375 |
| RPMI-8226 | 8.98 |
| SR | 0.562 |
| Non-small cell lung cancer | |
| A549/ATCC | 1.49 |
| EKVX | 2.39 |
| HOP-62 | 1.25 |
| HOP-92 | 2.11 |
| NCI-H23 | 1.36 |
| NCI-H322M | 2.13 |
| NCI-H460 | 1.48 |
| NCI-H522 | 0.238 |
| Colon cancer | |
| COLO 205 | 2.99 |
| HCC-2998 | 4.03 |
| HCT-116 | 0.495 |
| HCT-15 | >10 |
| HT29 | 1.34 |
| KM12 | 1.78 |
| SW-620 | 2.36 |
| CNS cancer | |
| SF-268 | 0.212 |
| SF-295 | 2.64 |
| SF-539 | 1.24 |
| SNB-19 | 2.59 |
| SNB-75 | 0.513 |
| U251 | 0.719 |
| Melanoma | |
| LOX IMVT | 0.577 |
| M14 | 1.48 |
| SK-MEL-2 | 2.11 |
| SK-MEL-28 | 2.21 |
| SK-MEL-5 | 1.11 |
| UACC-257 | 2.46 |
| UACC-62 | 0.419 |
| Ovarian cancer | |
| IGROV1 | 1.01 |
| OVCAR-3 | 3.51 |
| OVCAR-8 | 1.60 |
| SK-OV-3 | 4.33 |
| Renal cancer | |
| 786-0 | 1.66 |
| A498 | 3.95 |
| ACHN | 1.28 |
| CAKI-1 | 4.27 |
| SN12C | 3.01 |
| TK-10 | 3.13 |
| UO-31 | 4.05 |
| Prostate cancer | |
| PC-3 | 2.03 |
| DU-145 | 0.982 |
| Breast cancer | |
| MCF7 | 0.476 |
| NCI/ADR-RES | 8.56 |
| MDA-MB-231/ATCC | 5.97 |
| HS 578T | 1.63 |
| MDA-MB-435 | 1.86 |
| MDA-N | 2.22 |
| BT-549 | 3.29 |
| T-47D | 1.68 |

Assays were performed by NCI using the SRB method (48-h incubation).

Methotrexate-HSA Conjugate

To avoid systemic toxicity of methotrexate (MTX), a clinically used anticancer drug, and to improve tumour selectivity, MTX was conjugated to HSA forming a methotrexate-HSA conjugate (MTX-HSA) (Wosikowski et al., Clin. Cancer Res. 2003, 9, 1917-1926). MTX-HSA accumulates in tumor tissue due to the EPR effect and enters cells by endocytosis. Free MTX is released from MTX-HSA by lysosomal processes and went into cytosol, where it binds to its target enzyme dihydrofolate reductase, leading to tumor growth inhibition.

The concentration of MTX-HSA in tumor tissue was measured using radio-labelled MTX-HSA. MTX-HSA was given to female rates bearing the Walker-256 carcinoma at a dose of 13.2 μmol/kg MTX-HSA, which corresponds to 6 mg/kg of MTX (Wosikowski et al., Clin. Cancer Res. 2003, 9, 1917-1926). At 1 h after administration, the MTX-HSA concentration measured was 25 nmol/g tumor tissue, which is approximately 25 μM. At 3 h, the concentration in the tumor reached its maximum (29 nmol/g tumor tissue), which is approximately 29 μM. At 8 and 48 h, 19 μM and 18 μM of MTX-HSA, respectively, were measured. These results show that MTX-HSA is trapped in the tumor tissue for up to 48 h at concentrations between 18 and 29 μM, which are effective antiproliferative concentrations as determined in vitro.

The therapeutic efficacy of MTX and MTX-HSA was investigated in vivo in different human tumor xenografts growing s.c. in nude mice (Wosikowski et al., Clin. Cancer Res. 2003, 9, 1917-1926). MTX-HSA induced dose-dependent antitumor activity in vivo. When equivalent MTX doses were administered, superiority of MTX-HSA over MTX was observed.

MTX-HSA has been tested in clinical trials in humans. In a Phase I study of 17 patients, MTX-HSA was given at doses of 20, 40, 50, and 60 mg/m² MTX-HSA (based on the amount of MTX bound to albumin) (Hartung et al., Clin. Cancer Res. 1999, 5, 753-759). Mild anemia, transaminitis, and one case of skin toxicity were found. No significant leukopenia, nausea, renal toxicity, or other toxicities were observed. MTX-HSA was well tolerated. The half-life of the drug was estimated to be up to 3 weeks. In contrast, the half-life of the free MTX is approximately 7 h in human. Tumor responses were seen in three patients. A partial response was seen in one patient with renal cell carcinoma (response duration, 30 months, ongoing); a minor response was seen in one patient with pleural mesothelioma (response duration, 31 months, ongoing); and a minor response was seen in one patient with renal cell carcinoma (response duration, 14 months until progression).

In a Phase II study of 17 patients with metastatic renal cell carcinoma who progressed after first-line immunotherapy (Vis, et al., Cancer Chemother. Pharmacol. 2002, 49, 342-345), MTX-HSA was given once a week intravenously on an outpatient basis at a dose of 50 mg/m². Toxicity was manageable, relatively mild to moderate and reversible in most cases. Eight patients had stable disease (stabilization>2 months) for up to 8 months (median 121 days). However, no objective responses were seen. Another Phase II study (29 patients) was conducted in patients with malignant mesothelioma of the pleura or peritoneum by weekly i.v. infusion of 50 mg/m² (Max et al., Proceedings of American Society of Clinical Oncology, 2002). Weekly MTX-HSA was generally well tolerated with 2% grade 3 and 12% grade 4 thrombopenia and 10% grade 3 and 2% grade 4 stomatitis occurring after a mean treatment time of 6 weeks. All toxic side effects resolved spontaneously. Up to the reported date, 23/29 patients underwent at least one tumor assessment after 8-12 weeks demonstrating 1 PR and 1 MR (minor response) and 11 patients with NC>3 month while 6 patients experienced early disease progression within the first 2 months. Time to progression is in the range of 0.7 to 6.2+ months (mean 3.5 months, median 4.3 months). In conclusion, weekly MTX-HSA is a well-tolerated outpatient treatment for patients with advanced malignant mesothelioma, and antitumor activity had been demonstrated.

In situ Formation of Anticancer Drug-HSA Conjugates

Although the pharmaceutical applications of HSA have been evaluated intensively in the past, due to a variety of technical problems, only a handful of products were actually successful in reaching the market. One of the problems is that HSA is still obtained by conventional techniques involving the fractionation of plasma obtained from blood donors, which has the risk of transmitting possible viral/prion contaminants. To overcome this problem, Kratz et al. (J. Med Chem. 2000, 43, 1253-1256) proposed a new strategy making anticancer drug-HSA conjugate in situ. In this strategy, a thio-binding moiety is added to a drug, which binds to the cysteine-34 position of the circulating albumin after intravenous administration. The drug-HSA conjugate is selectively accumulated in tumors and releases the toxic free drug slowly at the tumor site.

HSA is a single-chain 66-kDa protein, which is largely a-helical and consists of three structurally homologous domains, organized into a heart shape (Carter and Ho, Adv. Protein Chem. 1994, 45, 153-203). HSA contains 17 disulfide bonds and one free thiol at cysteine-34. Approximately 70% of circulating albumin in the blood stream contains an accessible cysteine-34 which is not blocked by endogeneous sulfhydryl (HS) compounds such as cysteine, homocysteine, glutathione, and nitric oxide i.e., non-mercaptalbumin (Sogami et al., J. Chromatogr. 1985, 332, 19-27; Era et al., Int. J. Pept. Protein Res. 1988, 31, 435-442; Etoh, et al., J. Chromatogr. 1992, 578, 292-296). The free thiol group of cysteine-34 of HSA is an unusual feature of an extracellular protein. Only three other major proteins that contain cysteine residues that are not present as inter-chain disulfides occur in human plasma: apolipoprotein B-100 of low-density lipoprotein (LDL), which has two cysteine residues (cysteine-3734 and cysteine-4190) located at the C-terminal end of the protein (Coleman et al., Biochim. Biophys. Acta. 1990, 1037, 129-132; Yang, et al., Proc. Natl. Acad. Sci. 1990, 87, 5523-5527), fibronectin, which has two crytic free sulfhydryl groups (Smith et al., J. Biol. Chem. 1982, 260, 5831-583; Narasimhan and Lai, Biopolymers 1991, 31, 1159-1170), and α1-antitrypsin, which has a single cysteine residue (cysteine-232) (Shimokawa et al., J. Biochem. 1986, 100, 563-570; Morii, et al., J. Biochem. 1978, 83, 269-277). The thiol groups of these proteins do not react readily with sulfhydryl reagents under physiological conditions and are normally linked to either cysteine or glutathione in the blood circulation (Morii, et al., J. Biochem. 1978, 83, 269-277; Smith et al., J. Biol. Chem. 1982, 260, 5831-583; Shimokawa et al., J. Biochem. 1986, 100, 563-570; Coleman et al., Biochim. Biophys. Acta. 1990, 1037, 129-132; Ferguson, et al., Arch. Biochem. Biophys. 1997, 341, 287-294; Yang, et al., Proc. Natl. Acad. Sci. 1990, 87, 5523-5527).

The concentration of low molecular weight sulfhydryl compounds in human plasma in their reduced form, i.e., cysteine (˜10-12 uM) (Mansoor, et al., Anal. Biochem. 1992, 200, 218-229; Müller, et al., Am. J. Clin. Nutri. 1996, 63, 242-248), homocysteine (˜0.15-0.25 uM) (Mansoor, et al., Anal. Biochem. 1992, 200, 218-229; Müller, et al., Am. J. Clin. Nutri. 1996, 63, 242-248), cysteinylglycine (˜3-4 uM) (Mansoor, et al., Anal. Biochem. 1992, 200, 218-229; Müller, et al., Am. J. Clin. Nutri. 1996, 63, 242-248; Hagenfeldt et al., Clin. Chim. Acta 1978, 85, 167-173; Martensson, Metabolism 1986, 35, 118-121) or glutathione (˜4-5 uM) (Mansoor, et al., Anal. Biochem. 1992, 200, 218-229; Müller, et al., Am. J. Clin. Nutri. 1996, 63, 242-248; Martensson, Metabolism 1986, 35, 118-121), is low when compared to the total thiol concentration in human plasma which is in the range of 400-500 uM according to the literature (Hulea, et al., J. Enviro. Pathol. Toxicol. Oncol. 1995, 14, 173-180; Hack, et al., Blood 1998, 92, 59-67). The free thiol group of the HSA's cysteine-34 accounts for the majority of the total thiols (80-90%) in the blood plasma. In addition, the HS group of the HSA's cysteine-34 is the most reactive thiol group in human plasma because of the low pKₐ of cysteine-34 (approximately 7) in HSA, compared to that of cysteine (8.5) and glutathione (8.9) (Pedersen and Jacobsen, Eur J Biochem. 1980, 106, 291-5). In summary, the free HS group of the HSA's cysteine-34 is a unique and accessible functional group of a plasma protein that can be exploited for in situ coupling with a thiol-reactive anticancer drug to form a prodrug after intravenous administration. This strategy has been employed making the anticancer drug-HSA conjugates (Kratz et al., Drug Delivery 1998, 5, 1-19; Kratz et al., J. Med. Chem. 2000, 43, 1253-1256; Kratz et al., J. Med. Chem. 2002, 45, 5523-5533; Warnecke and Kratz, Bioconjug. Chem. 2003, 14, 377-387).

Albumin-binding doxorubicin (Dox) prodrugs have been synthesized (Kratz et al., J. Med. Chem. 2002, 45, 5523-5533). These prodrugs, especially the (6-maleimidocaproyl)hydrazone derivative (Dox-MH), are rapidly and selectively bound to the free HS groups of the HSA's cysteine-34 when incubated with endogenous albumin. Dox-MH was distinctly superior to free doxorubicin in three animal tumor models (mouse renal RENCA, human breast cancers MDA-MB 435 and MCF-7) with respect to antitumor efficacy and toxicity.

When Dox-MH was incubated with exogenously added HSA, majority of the Dox-MH was reacted with HSA within 5 min (Kratz et al., J. Med. Chem. 2002, 45, 5523-5533). By 90 min, all of the Dox-MH had reacted. To determine the coupling rate and selectivity of Dox-MH for endogenous albumin, Dox-MH was incubated with human blood plasma (Kratz et al., J. Med. Chem. 2002, 45, 5523-5533). The reaction of Dox-MH with HSA was almost complete after 2 min. Once again, by 90 min, all of the Dox-MH had reacted. These data demonstrate that Dox-MH reacts with HSA efficiently.

Dox-MH was tested in nude mice bearing the MDA-MB 435 human breast cancer. At an optimal dose of 3×39.3 umol/kg (3×23 mg/kg), complete remissions were achieved. In contrast, free Dox only showed modest antitumor activity. Most importantly, in addition to producing tumor remissions, treatment with Dox-MH did not produce an overall change in body weight after 24 days of drug administration. In contrast, treatment with free Dox produced a significant body weight loss (about 10%), suggesting that Dox-MH has a greater therapeutic index than Dox.

CC-1065 Analogue-HSA Conjugates

The poor therapeutic efficacy of most anticancer drugs is caused by two major problems. One is the lack of tumor-specificity, i.e. the drugs are not preferentially accumulated by tumors; and the other is the short half-life of the drug. Albumin-drug conjugates overcome these two problems. In most cases, a high degree of binding to albumin is a disadvantage because it reduces the amount of drugs available. However, the in situ formation of albumin prodrugs turns this disadvantage into a therapeutic advantage if a cleavable bond between the drug and the albumin is employed. The albumin prodrugs are preferentially accumulated in tumor tissues and have longer half-lives, leading to an improved therapeutic index. Experimental and clinical data with MTX-HSA demonstrated that the conjugate was preferentially accumulated in tumors and had a very long half-life. These improved drug properties led to an improved clinical efficacy. For the same reasons, Dox-MH had a better antitumor efficacy than free Dox.

In MTX-HSA, MTX is first mixed with albumin ex vivo, and then given to patients. One of the problems associated the use of this approach is that HSA has to be obtained. Today HSA is still obtained by conventional techniques involving the fractionation of plasma obtained from blood donors, which has the risk of transmitting possible viral/prion contaminants. Further, because MTX is conjugated to HSA ex vivo, MTX may break off from the conjugate during the storage and transportation, compromising the tumor-targeting strategy. Last, but not the least that this approach is inconvenient and adds extra cost to the therapy. In Dox-MH, the use of albumin ex vivo is avoided; however, Dox is not potent enough, and a large amount of drugs has to be used. In addition, in Dox-MH, a very labile hydrazone bond is used. The hydrazone bond is easily broken during circulation before the conjugate is taken up by tumor cells. When free drug is released prematurely, the efficacy of the conjugate is compromised. For these reasons, there is a need to find intensely potent cytotoxic agents and linkers that are suitable for use in targeting albumin to treat cancer.

The CC-1065 class of compounds has distant advantages over other compounds that use targeting albumin for cancer chemotherapy. First, because the CC-1065 class of compounds is S-phase-specific, a prolonged exposure to cancer cells is critical for achieving an optimal efficacy, and conversely, toxic side effects are minimized. Second, because this CC-1065 class of compounds is extremely potent, little of the drug is used. When given to patients, most of the CC-1065 or analog will react with HSA forming an HSA-prodrug, resulting in an increase in anticancer efficacy and a decrease in toxic side effects. In fact, this these CC-1065 class conjugated compounds can be used where other anticancer drugs cannot since most anticancer drugs are not potent enough. For example, if a large quantity of a particular anticancer drug has to be given to a patient, there is probably not enough HSA in the blood to which it can form conjugates. As a result, the drug will remain as free (unbound) drug, and the advantages of conjugation such as long residence time, lowered toxicity, and increased efficacy as to the tumor will not be observed. In many cases where free drugs are conjugated, the conjugation chemistry consumes a large enough portion of albumin that it affects albumin's normal biological functions. As such, treatment with such drugs will cause severe side effects in part from the alteration of albumin. For these reasons, the CC-1065 class of compounds, and preferably the analogs described herein, represent an important class of anticancer drugs that will show greatly improved therapeutic efficacy when used with albumin targeting/conjugation strategies.

Accordingly, the present invention provides compounds having generally the formula:
CC-1065 analogue-linker-maleimide (Formula I)
wherein:
the linker is selected from —C(O)R₁—, —C(O)OR₁—, —C(O)NR₂R₃—,
   —C(O)(CH₂)ₙ₁(OCH₂CH₂)ₙ₂— where ₙ₁ is 1-6, and where ₙ₂ is 0-20,
   —C(O)(CH₂)ₙ₃R₄(CH₂)ₙ₄— where ₙ₃ and ₙ₄ are independently 0-10,
   —C(O)(CH₂)ₙ₁R₄(OCH₂CH₂)ₙ₂— where ₙ₁ is 1-6, and ₙ₂ is 0-20,
   —C(O)(CH₂)ₙ₁(OCH₂CH₂)ₙ₂R₄— where ₙ₁ is 1-6, and ₙ₂ is 0-20,
   —C(O)NR₂R₃(CH₂)ₙ₃R₄(CH₂)ₙ₄— where ₙ₃ and ₙ₄ are independently 0-10 or
   —C(O)NR₂R₃(CH₂)ₙ₅(OCH₂CH₂)ₙ₆— where ₙ₅ and ₙ₆ are independently 0-10;
wherein:
R₁ is alkyl or aryl;
R₂ and R₃ are independently H, alkyl or aryl but R₂ and R₃ cannot be aryl at the same time;
R₄ is a valence bond, aryl, or alkyl and containing at least one nitrogen; and
   the CC-1065 analogue is a compound generally having the following structure (Formula II): 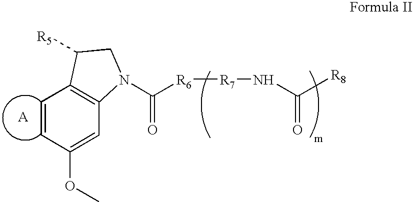
   wherein:
A is a 5-6 member ring such as alkyl, aryl or heteroaryl;
R₅ is CH₂Cl, CH₂Br, CH₂I or CH₂OSO₂CH₃;
R₆ is a valence bond, a C₁-C₆ alkyl, a C₂-C₆ alkenyl, a C₂-C₆ alkynyl or an aryl;
R₇ and R₈ are independently aryl or heteroaryl; and
M is 0-2; and
   maleimide, generally having the structure given by formula III.

Maleimide is a structure of formula III:

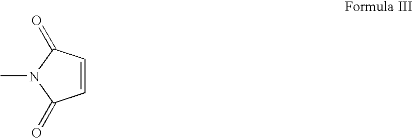

In preferred embodiments the CC-1065 analogue is a compound generally having the structure given as formula IV, V or VI: 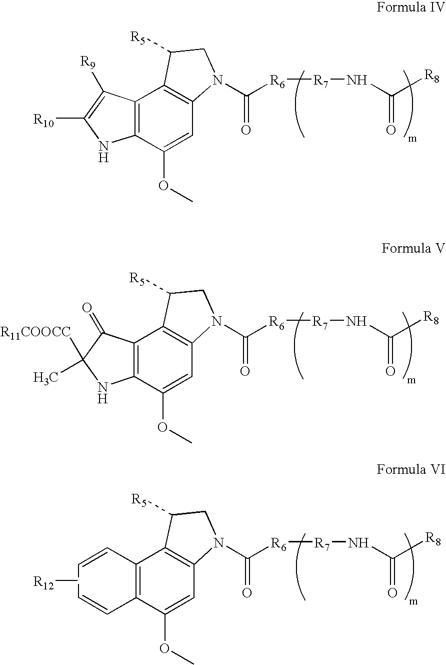
wherein:
R₅ is CH₂Cl, CH₂Br, CH₂I or CH₂OSO₂CH₃;
R₆ is a valence bond, a C₁-C₆ alkyl, a C₂-C₆ alkenyl, a C₂-C₆ alkynyl or an aryl;
R₇ and R₈ are independently aryl or heteroaryl;
M is 0-2;
R₉ is H, C₂-C₆ alkyl, C(O)-alkyl, C(O)O-alkyl;
R₁₀ is H, C₂-C₆ alkyl;
R₁₁ is CH₃ or CF₃; and
R₁₂ is H, NH₂, NO₂, O-alkyl, NH-alkyl, N(alkyl)₂, NHC(O)-alkyl, ONO₂, F, Cl, Br, I, OH, OCF₃, OSO₂CH₃, CO₂H, CO₂-alkyl, CO₂CF₃ or CN;

The linker is generally selected from the group including —C(O)R—, —C(O)OR₁—, —C(O)NR₂R₃—, —C(O)(CH₂)ₙ₁(OCH₂CH₂)ₙ₂— where ₙ₁ is 1-6, and ₙ₂ is 0-20,
—C(O)(CH₂)ₙ₃R₄(CH₂)ₙ₄— where ₙ₃ and ₙ₄ are independently 0-10,
—C(O)(CH₂)ₙ₁R₄(OCH₂CH₂)ₙ₂— where ₙ₁ is 1-6, and ₙ₂ is 0-20,
—C(O)(CH₂)ₙ₁(OCH₂CH₂)ₙ₂R₄— where ₙ₁ is 1-6, and ₙ₂ is 0-20,
—C(O)NR₂R₃(CH₂)ₙ₃R₄(CH₂)ₙ₄— where ₙ₃ and ₙ₄ are independently 0-10, and
—C(O)NR₂R₃(CH₂)ₙ₅(OCH₂CH₂)ₙ₆— where ₙ₅ and ₙ₆ are independently 0-10.

Other preferred embodiments of the compounds include those where the CC-1065 analogue is a compound of formula VI: 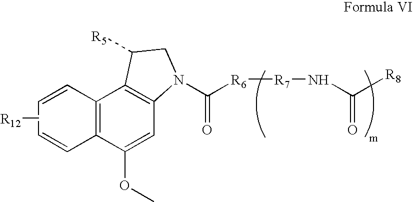
wherein:
The linker is —C(O)R₁— and R₁ is alkyl;
R₅ is CH₂Cl, CH₂Br, CH₂I or CH₂OSO₂CH₃;
R₆ is a valence bond or CH═CH;
R₇ and R₈ are independently heteroaryl; and
R₁₂ is H; and
   Maleimide.

A preferred antiproliferative compound of the present invention is that shown below as (+)-YW-391:

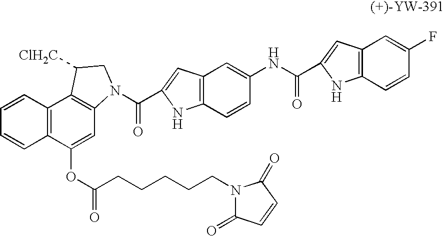

Another preferred antiproliferative compound of the present invention is that shown below as (+)-YW-392:

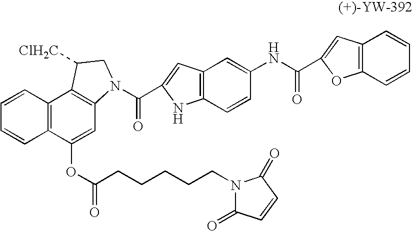

The compounds of the present invention are effective for the treatment of mammalian diseases characterized by aberrant cellular proliferation, such as tumors, cancer, leukemias, autoimmune diseases, and the like. The compounds of the present invention have affinity for albumin, which confers an increased half-life in vivo, and allows them to be accumulated by proliferating mammalian cells, e.g., tumor cells. As a result of their increased half-life in vivo and proliferating cell-selective accumulation, e.g., tumor-selective accumulation, these compounds are more effective than traditional therapies in treating cancer, and have decreased toxic side effects.

Formulations and Methods of Treatment

The antiproliferative compounds can be prepared as pharmaceutical formulations, suitable for administration to human subjects afflicted with diseases characterized by aberrant cellular proliferation. Such pharmaceutical formulations include salts of the compounds, albumin conjugates of the compounds, as well as formulations having appropriate excipients. In a pharmaceutical formulation, the antiproliferative compounds described herein are referred to hereinafter as “active compounds” or “actives” or alternatively as “drugs” within the formulation. In a combination therapy, other antiproliferative agents may be included, in which case the formulation would include more than one active agent, e.g., an exemplary pharmaceutical formulation of cisplatinum and (+)-YW-392 (two active agents) in saline (an excipient) suitable for intravenous administration to a human patient.

Compositions intended for oral use may be prepared according to any method known to the art for the manufacture of pharmaceutical compositions and such compositions may contain one or more compounds selected from the group consisting of sweetening compounds, flavoring compounds, coloring compounds and preserving compounds in order to provide pharmaceutically elegant and palatable preparations. Tablets contain the active compound in a mixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating compounds, for example, corn starch, or alginic acid; binding compounds, for example starch, gelatin or acacia, and lubricating compounds, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active material in admixture with excipients suitable for the manufacture of aqueous suspensions. Such excipients are suspending compounds, for example sodium carboxymethylcellulose, methylcellulose, hydropropylmethylcellulose, sodium alginate, polyvinylpyrrolidone, gum tragacanth and gum acacia; dispersing or wetting compounds may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring compounds, one or more flavoring compounds, and one or more sweetening compounds, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening compound, for example beeswax, hard paraffin or acetyl alcohol. Sweetening compounds such as those set forth above, and flavoring compounds may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting compound, suspending compound and one or more preservatives. Suitable dispersing or wetting compounds and suspending compounds are exemplified by those already mentioned above. Additional excipients, for example sweetening, flavoring and coloring compounds, may also be present.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying compounds may be naturally occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example sweetening, flavoring and coloring compounds, may also be present.

Syrups and elixirs may be formulated with sweetening compounds, for example glycerol, propylene glycol, sorbitol or sucrose. Such formulations may also contain a demulcent, a preservative and flavoring and coloring compounds. The pharmaceutical compositions may be in the form of a sterile injectable aqueous or oleaginous suspension. This suspension may be formulated according to the known art using those suitable dispersing or wetting compounds and suspending compounds which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The active compound may also be administered in the form of suppositories for rectal administration of the drug. These compositions can be prepared by mixing the drug with a suitable non-irritating excipient, which is solid at ordinary temperatures but liquid at the rectal temperature and will therefore melt in the rectum to release the drug. Such materials are cocoa butter and polyethylene glycols.

The active compound may be administered parenterally in a sterile medium. The drug, depending on the vehicle and concentration used can either be suspended or dissolved in the vehicle. Advantageously, adjuvants such as local anesthetics, preservatives and buffering compounds can be dissolved in the vehicle.

Compositions of the present invention (i.e., the albumin conjugates) may be administered continuously or intermittently by any route which is compatible with the particular molecules. Thus, as appropriate, administration may be oral or parenteral, including subcutaneous, intravenous, inhalation, nasal, and intraperitoneal routes of administration. In addition, intermittent administration may be by periodic injections of a bolus of the composition once daily, once every two days, once every three days, once weekly, twice weekly, biweekly, twice monthly, and monthly.

Therapeutic compositions of the present invention may be provided to an individual by any suitable means, directly (e.g., locally, as by injection, implantation or topical administration to a tissue locus) or systemically (e.g., parenterally or orally). Where the composition is to be provided parenterally, such as by intravenous, subcutaneous, intramolecular, ophthalmic, intraperitoneal, intramuscular, buccal, rectal, vaginal, intraorbital, intradermal, transdermal, intratracheal, intracerebral, intracranial, intraspinal, intraventricular, intrathecal, intracistemal, intracapsular, intranasal or by aerosol administration, the composition preferably comprises part of an aqueous or physiologically compatible fluid suspension or solution. Thus, the carrier or vehicle is physiologically acceptable so that in addition to delivery of the desired composition to the patient, it does not otherwise adversely affect the patient's electrolyte and/or volume balance. The fluid medium for the agent thus can comprise normal physiologic saline (e.g., 0.9% aqueous NaCl) or a buffer, pH 3-7.4. Alternatively, the use of continuous or pulsatile administration of the therapeutic compositions of the present invention by mini-pump can be employed in the methods of the present invention.

Useful solutions for parenteral administration may be prepared by any of the methods well known in the pharmaceutical art, described, for example, in REMINGTON'S PHARMACEUTICAL SCIENCES (Gennaro, A., ed.), Mack Pub., 1990. Formulations of the therapeutic agents of the invention may include, for example, polyalkylene glycols such as polyethylene glycol, oils of vegetable origin, hydrogenated naphthalenes, and the like. Formulations for direct administration, in particular, may include glycerol and other compositions of high viscosity to help maintain the agent at the desired locus. Biocompatible, preferably bioresorbable, polymers, including, for example, hyaluronic acid, collagen, tricalcium phosphate, polybutyrate, lactide, and glycolide polymers and lactide/glycolide copolymers, may be useful excipients to control the release of the agent in vivo. Other potentially useful parenteral delivery systems for these agents include ethylene-vinyl acetate copolymer particles, osmotic pumps, implantable infusion systems, and liposomes. Formulations for inhalation administration contain as excipients, for example, lactose, or may be aqueous solutions containing, for example, polyoxyethylene-9-lauryl ether, glycocholate and deoxycholate, or oily solutions for administration in the form of nasal drops, or as a gel to be applied intranasally. Formulations for parenteral administration may also include glycocholate for buccal administration, methoxysalicylate for rectal administration, or cutric acid for vaginal administration. Suppositories for rectal administration may also be prepared by mixing the therapeutic compositions of the present invention (alone or in combination with a chemotherapeutic agent) with a non-irritating excipient such as cocoa butter or other compositions that are solid at room temperature and liquid at body temperatures.

Where the compound provided by the present invention is given by injection, it can be formulated by dissolving, suspending or emulsifying it in an aqueous or nonaqueous solvent. Methyl sulfoxide, N,N-dimethylacetamide, N,N-dimethylformamide, vegetable or similar oils, synthetic aliphatic acid glycerides, esters of higher aliphatic acids and proylene glycol are examples of nonaqueous solvents. The compound is preferably formulated in aqueous solutions such as Hank's solution, Ringer's solution or physiological saline buffer.

Where compound provided by the present invention is given orally, it can be formulated through combination with pharmaceutically acceptable carriers that are well known in the art. The carriers enable the compound to be formulated, for example, as a tablet, pill, suspension, liquid or gel for oral ingestion by the patient. Oral use formulations can be obtained in a variety of ways, including mixing the compound with a solid excipient, optionally grinding the resulting mixture, adding suitable auxiliaries and processing the granule mixture. The following list includes examples of excipients that can be used in an oral formulation: sugars such as lactose, sucrose, mannitol or sorbitol; cellulose preparations such as maize starch, wheat starch, potato starch, gelatin, gum tragacanth, methyl cellulose, hydroxyproylmethyl-cellulose, sodium carboxymethylcellulose and polyvinylpyrrolidone (PVP).

The compounds of the present invention can also be delivered in an aerosol spray preparation from a pressurized pack, a nebulizer or from a dry powder inhaler. Suitable propellants that can be used in a nebulizer include, for example, dichlorodifluoro-methane, trichlorofluoromethane, dichlorotetrafluoroethane and carbon dioxide. The dosage can be determined by providing a valve to deliver a regulated amount of the compound in the case of a pressurized aerosol.

Formulations for topical administration to the skin surface may be prepared by dispersing the molecule capable of releasing the therapeutic compositions of the present invention (alone or in combination with a chemotherapeutic agent) with a dermatologically acceptable carrier such as a lotion, cream, ointment or soap. Particularly useful are carriers capable of forming a film or layer over the skin to localize application and inhibit removal. For topical, administration to internal tissue surfaces, the agent may be dispersed in a liquid tissue adhesive or other substance known to enhance adsorption to a tissue surface. For example, hydroxypropylcellulose or fibrinogen/thrombin solutions may be used to advantage. Alternatively, tissue-coating solutions, such as pectin-containing formulations may be used.

The compounds of the present invention can be used in the treatment of cancer and other mammalian cell proliferating diseases. The compounds of the present invention can be provided simultaneously or sequentially in time. The compounds of the present invention can be administered alone or in combination with other therapeutic agents, e.g., chemotherapeutic compounds.

Pharmaceutical compositions of the present invention contain a therapeutically effective amount of the compound provided by the present invention. The amount of the compound will depend on the patient being treated as well as the particular disorder. The patient's weight, severity of illness, manner of administration, cotherapies and judgment of the prescribing physician should be taken into account in deciding the proper dosages. The determination of a therapeutically effective amount of a compound is well within the capabilities of one with skill in the art.

Although a therapeutically effective amount of a compound provided by the present invention will vary according to the patient being treated, suitable doses will typically be in the range between about 0.1 microgram/kg/day and 10 mg/kg/day of the compound. More preferably, suitable dosage ranges include 0.5 micrograms/kg to 5 mg/kg per day. Even more preferably, the compounds are given at dose ranges from 1 micrograms/kg/day to 1 mg/kg/day, and most preferably at 1 micrograms/kg to 100 micrograms/kg per day. Specific doses for humans can be calculated or extrapolated from the IC₅₀ values provided in the examples.

In some cases, it may be necessary to use dosages outside of the stated ranges to treat a patient, e.g. smaller doses with combination therapy or larger doses for heroic measures. Those cases will be apparent to the prescribing physician. Where it is necessary, a physician will also know how and when to interrupt, adjust or terminate treatment in conjunction with a response of a particular patient.

The invention is further defined by reference to the following examples, which are not meant to limit the scope of the present invention. It will be apparent to those skilled in the art that many modifications, both to the materials and to methods, may be practiced without departing from the purpose and interest of the invention. Compounds of the present invention may be tested for efficacy in vitro and in experimental animal tumor models using the assays described below; an effective compound will inhibit tumor growth both in vitro and in experimental animal tumor models. Compounds most preferred in the invention are those that have the greatest antitumor effects in experimental animal tumor models.

### EXAMPLE 1

### Synthesis of (+)-YW-367

Anhydrous HCl in ethyl acetate (3 N, 2 mL) was added to (+)-CBI (20 mg, 0.1 mmol), and the reaction mixture was stirred for 30 min at room temperature in the dark (FIG. 2). Solvent was removed. DMF (1 mL) was added, followed by the addition of 5-[(5-fluoro-1H-indol-2-ylcarbonyl)amino]-1H-indol-2-carboxylic acid (34 mg, 0.1 mmol) and EDCI (64 mg). The reaction mixture was stirred overnight at room temperature. The product was purified by thin layer chromatography, eluting with ethyl acetate to afford (+)-YW-367 as a gray powder (55% yield). ¹H NMR (DMSO-d6, ppm): 11.86 (s, 1 H, NH), 11.76 (s, 1 H, NH), 10.45 (s, 1 H, OH), 10.23 (s, 1 H, NH), 8.23-7.08 (m, 13 H, Ar—H), 4.85 (t, 1 H, J=10.8 Hz, NCHH), 4.55 (dd, 1 H, J=2.0 Hz, 11.2 Hz, NCHH), 4.24 (m, 1 H, ClCH₂CHCH₂), 4.03 (d, 1 H, J=10.5 Hz, CHHCl), 3.89 (dd, 1 H, J=7.2, 11.19 Hz, CHHCl). HRMS (EI) calcd for (C₃₁H₂₂ClFN₄O₃) 551.1286, found 551.1279.

### EXAMPLE 2

### Synthesis of (+)-YW-391

(+)-YW-391 was synthesized by esterification of (+)-YW-367 with 6-maleimidocaproic acid (FIG. 3). Briefly, to acetonitrile (1.6 mL) was added (+)-YW-367 (19 mg, 0.034 mmol), 6-maleimidocaproic acid (22 mg, 0.103 mmol), 2-(1H-benzotriazol-1-yl)-1,1,3,3-tetramethyluronium hexafluorophosphate (HBTU, 17 mg, 0.045 mmol) and diisopropylethylamine (0.045 mL). The reaction mixture was allowed to proceed overnight. The product was extracted with ethyl acetate, and the organic phase was washed with water. The solution was dried by anhydrous sodium sulfate, and the solution was filtered. Solvent was removed in vacuo, and the product was purified by thin layer chromatography to afford 10 mg (26% yield) of (+)-YW-391 as an off white powder. ¹H NMR (DMSO-d6, ppm): 11.84(s, 1H, NH), 11.76 (s, 1H, NH), 10.23 (s, 1H, NH), 8.24-7.06 (m, 13H), 7.03 (s, 2H), 4.97-4.90 (t, 1H, J=10.79 Hz, NCHH), 4.68-4.65 (dd, 1H, J=2.40, 11.19 Hz, NCHH), 4.50-4.40 (m, 1H, ClCH₂CHCH₂), 4.13-4.09 (dd, 1H, CHHCl), 4.06-4.01 (dd, 1H, J=6.80, 11.59 Hz, CHHCl), 3.45 (t, 2H, J=13.99 Hz, CH₂), 2.83 (t, 2H, J=14.79, CH₂), 1.77-1.71 (m, 2H, CH₂), 1.62-1.56 (m, 2H, CH₂), 1.42-1.36 (m, 2H, CH₂). MS (M+H): 746.

### EXAMPLE 3

### Synthesis of (+)-YW-392

(+)-YW-392 was synthesized by a procedure similar to that for synthesis of (+)-YW-391 (FIG. 4). ¹H NMR (DMSO-d6, ppm): 11.77 (s, 1H, NH), 10.49 (s, 1H, NH), 8.25-7.28 (m, 13H), 7.03 (s, 2H), 4.95-4.90 (t, 1H, J=10.79 Hz, NCHH), 4.68-4.65 (dd, 1H, J=2.40, 11.19 Hz, NCHH), 4.46-4.42 (m, 1H, ClCH₂CHCH₂), 4.13-4.09 (dd, 1H, CHHCl), 4.06-4.01 (dd, 1H, J=6.80, 11.59 Hz, CHHCl), 3.45 (t, 2H, J=13.99 Hz, CH₂), 2.83 (t, 2H, J=14.79, CH₂), 1.78-1.71 (m, 2H, CH₂), 1.63-1.56 (m, 2H, CH₂), 1.42-1.36 (m, 2H, CH₂). MS (M+H): 729.

### EXAMPLE 4

### Mechanism of Action—Apoptosis

The mechanism of antitumor activity of the newly synthesized analogues was studied in U937 cells using (±)-YW-201, possessing a similar chemical structure to (±)-YW-367. At a 10 nM concentration, (±)-YW-201 caused DNA fragmentation in about 12%, 20%, 40% and 80% of U937 cells, respectively, following an incubation time of 2, 3, 4 and 6 h (FIG. 5).

### EXAMPLE 5

### Anticancer Activity Assay in vitro

The antitumor activity of the compounds was evaluated in vitro against L1210 leukemia and human SKOV-3 ovarian cancer cells (Table 4). L1210 leukemia cells and SKOV-3 (2.5×10⁴ cells/well) in RPMI-1640 medium supplemented with 10% FCS medium were seeded in a 96-well plate. Drugs (10 uL) at increasing concentrations were added to each well, and the total volume was adjusted to 0.1 mL/well using the same medium. For L1210 leukemia cells, the plate was incubated for 24 h at 37° C. followed by addition of 10 uL of ³H-thymidine (20 uCi/mL). For SKOV-3 ovarian cancer cells, the plate was incubated for 48 h at 37° C. followed by addition of 10 uL of ³H-thymidine (20 uCi/mL). The plate was incubated for another 24 h. The cells were harvested, and radioactivity was counted using the Packard Matrix 96 beta counter. The results were expressed as the minimal concentration that inhibits ³H-thymidine incorporation by 50% (IC₅₀). Percent growth inhibition was calculated as follows: [(total cpm−experimental cpm)/total cpm]×100. (+)-YW-391 and (+)-YW-392 were very potent against both the L1210 leukemia and SKOV-3 human ovarian cancer cells. For example, IC₅₀ values are 0.17, 25 and 31 nM for (+)-YW-367, (+)-YW-391 and (+)-YW-392, respectively against L1210 leukemia cells. The IC₅₀ values are 14 and 24 nM, respectively, for (+)-YW-367 and (+)-YW-391 against SKOV-3 human ovarian cancer cells.

TABLE 4
Antitumor activity against cancer cells in vitro

IC₅₀ (nM)

| Drug | L1210^{a} | SKOV-3^{b} |
|---|---|---|
| (+)-YW-367 | 0.17 | 14 |
| (+)-YW-391 | 25 | 24 |
| (+)-YW-392 | 31 | — |

^{a}Cytotoxicity was measured in a 48-h proliferation assay;
^{b}Cytotoxicity was measured in a 72-h proliferation assay.
The results were reported as the minimal drug concentration that inhibits uptake of ³H-thymidine by 50%, and were the mean values of two experiments.

### EXAMPLE 6

### Anticancer Activity in the L1210 Leukemia Tumor Model

The compounds were tested in male BDF₁ mice (18-22 g, 6 mice/group) bearing L1210 leukemia cells. Tumor lines were propagated in DBA/2 female mice with cell (10⁵ cells/mouse) transfer every 7 days. Diluted ascitic fluid (0.1 mL) containing 10⁵ leukemia cells were inoculated, i.p. in mice on day 0. In this L1210 leukemia model, (+)-YW-391 was highly active against L1210 leukemia, and was active over a 8-fold dose range (0.05-0.4 mg/kg) (Table 5). At an optimal dose of 0.4 mg/kg, it produced an ILS of 133%. Most importantly, at optimal dose, (+)-YW-391 (ILS: 133%) had a much higher therapeutic efficacy than doxorubicin (Dox), which only produced an ILS of 85%. Dox is one of the best drugs against L1210 leukemia. In addition, no delayed toxicity and no other side effect except weight loss were noted within a 6-month observation period when the drug was given to no tumor-bearing mice (0.5 mg/kg, i.v. one dose).

TABLE 5
Antitumor activity against L1210 leukemia cells in mice*

| Drug | Dose (mg/kg) | % ILS |
|---|---|---|
| (+)-YW-391 | 0.4 | 133 |
| | 0.2 | 107 |
| | 0.1 | 51 |
| | 0.05 | 40 |
| Doxorubicin | 10.0 | 85 |

*Female BDF₁ mice (6/group) were injected i.p. with 10⁵ cells on day 0. Drugs were administrated i.v. on day 1. The median number of days of survival of the vehicle-treated mice was 7.5.

### EXAMPLE 7

### Antitumor Activity of (+)-YW-391 and (+)-YW-392 in Mice Bearing Colon 38 Adenocarcinoma

Both (+)-YW-391 and (+)-YW-391 were remarkably active against colon 38 adenocarcinoma in mice (Table 6). At the maximal tolerated dose (MTD) (0.3 mg×2), (+)-YW-391 cured 50% (3/6) of mice (tumor-free on day 60), and at 0.15 mg/kg, (+)-YW-391 cured 29% (2/7) of mice. (+)-YW-391 produced a tumor growth inhibition (TGI) of 99% at both the 0.3 and 0.13 mg/kg dose levels. In sharp contrast, at MTD, both 5-FU and CPT-11, two of the most effective drugs against colon cancer in the clinic, only produced a TGI of 95 and 96%, respectively, without any cures. At the lowest dose of 0.15 mg/kg (LCK: 2.3), (+)-YW-391 killed 10-times more tumor cells than 5-FU at a very toxic dose of 70 mg/kg (LCK: 1.1). (+)-YW-391 was also significantly better than cisplatin, one of the best drugs against colon cancer. Furthermore, (+)-YW-391 is remarkably better than adozelesin, bizelesin and carzelesin. These data strongly suggest that (+)-YW-391 has the potential to be a powerful new treatment for colon cancer. Furthermore, we noted that mice could tolerate a greater weight loss with (+)-YW-391 than with 5-FU or CPT-11. For example, with (+)-YW-391, the mice did not die with a weight loss of-25%. In contrast, with 5-FU and CPT-11, the mice begin to die when the weight loss reached -7%.

TABLE 6
Anticancer activity of CC-1065 compounds in mice bearing colon 38^{a}

| | Dose | | % Tumor growth | % Maximum | Log₁₀ cell | % Cured |
|---|---|---|---|---|---|---|
| Drug | (mg/kg) | Schedule | inhibition | weight loss | kill (LCK) | mice |
| YW-391 | 0.3 | q4d × 2 | 99 | −25 | 2.6 | 50 |
| | 0.15 | q4d × 3 | 99 | −10 | 2.3 | 29 |
| YW-392 | 0.3 | q4d × 3 | 90 | −7 | 1.7 | 0 |
| | 0.15 | q4d × 3 | 84 | −5 | — | 0 |
| 5-FU^{b} | 70 | q4d × 3 | 95 | −7 | 1.1 | 0 |
| CPT-11^{c} | 100 | q4d × 3 | 96 | −7 | 1.7 | 0 |
| Cisplatin | 4 | day1 | 66 | −15 | 0.48 | 0 |
| | 2 | q4d × 3 | 42 | −8 | 0.30 | 0 |
| Adozelesin^{d} | 0.05 | days 2, 9 | 93 | not reported | 0.65 | 0 |
| Bizelesin^{e} | 0.005 | q4d × 3 | not reported | not reported | 0.70 | 0 |
| Carzelesin^{f} | 0.2 | q4d × 3 | 92 | not reported | not reported | 0 |

^{a}Female BDF₁ mice were implanted s.c. with 10⁶ cells on day 0. All drugs were given i.v.;
^{b}4/6 mice died of drug toxicity;
^{c}1/6 mice died of drug toxicity;
^{d}From Li et al. Invest. New Drugs 1991, 9, 137-148, 1991;
^{e}From Carter et al., Clin. Cancer Res. 1996, 2, 1143-1149;
^{f}From Li et al., Invest. New Drugs 1991, 9, 137-148. For all of our tested drugs, P < 0.01 compared with non-drug treated animals.

### EXAMPLE 8

### Antitumor Activity in Mice Bearing JC Breast Adenocarcinoma (JC)

JC is an epithelial-like cell line established in 1983 from a spontaneous primary adenocarcinoma along the milk line (Capone et al., Cancer Immuno. Immunother. 1987, 25, 93-9). It produces tumors with papillary adenocarcinoma morphology in BALB/c mice. (+)-YW-391 was highly active against JC in mice with TGIs of 98% (FIG. 6 and Table 7). Most importantly, (+)-YW-391 was significantly more efficacious than Dox, one of the best drugs against breast cancer. For example, at MTD, while Dox (8 mg/kg) had a TGI of 82%, (+)-YW-391 had a TGI of 98%. At the lowest dose of 0.16 mg/kg, (+)-YW-391 (LCK: 1.5) killed 10-times more tumor cells than Dox at 8 mg/kg (LCK: 0.78). (+)-YW-391 has the potential to be a powerful new treatment for human breast cancer.

TABLE 7
Anticancer activity of CC-1065 compounds in mice bearing JC
breast cancer*

| | | | % Tumor | | |
|---|---|---|---|---|---|
| | Dose | | growth | Log₁₀ cell | % Maximum |
| Drug | (mg/kg) | Schedule | inhibition | kill (LCK) | weight loss |
| YW-391 | 0.25 | q4d × 2 | 98 | 2.0 | −21 |
| | 0.20 | q4d × 3 | 98 | 1.9 | −23 |
| | 0.16 | q4d × 3 | 93 | 1.5 | −19 |
| Doxorubicin | 8.0 | q4d × 3 | 82 | 0.78 | −25 |

*Female Balb/c mice (8/group) were implanted s.c. with 10⁶ cells on day 0. All drugs were given i.v. For all tested drugs, P < 0.01 compared with non-drug treated animals.

### EXAMPLE 9

### Antitumor Activity in Mice Bearing Lewis Lung Carcinoma

(+)-YW-391 was highly active in mice bearing Lewis lung carcinoma with TGIs of 98% (FIG. 7 and Table 8). Most importantly, (+)-YW-391 was significantly more efficacious than cisplatin, one of the best drugs against human lung cancer.

TABLE 8
Anticancer activity of YW-391 in mice bearing Lewis Lung Carcinoma*

| | | | % Tumor | | |
|---|---|---|---|---|---|
| | Dose | | growth | Log₁₀ cell | % Maximum |
| Drug | (mg/kg) | Schedule | inhibition | kill (LCK) | weight loss |
| YW-391 | 0.2 | 1, 5, 12 | 84 | 0.75 | −17 |
| Cisplatin | 3.0 | 1, 5, 12 | 74 | 0.45 | −13 |

*BDF₁ female mice (6/group) were implanted s.c. with 10⁶ cells on day 0. Drugs were given i.v. on days 1, 5 and 12. For all tested drugs, P < 0.01 compared with non-drug treated animals.

### EXAMPLE 10

### Antitumor Activity in SKOV-3 Human Ovarian Cancer Xenograft

Ovarian cancer is one of the most difficult to treat human cancers, and very few drugs are effective. YW-391 had significant activity in SKOV-3 human ovarian cancer xenograft (FIG. 8 and Table 9). In fact, YW-391 is more effective than paclitaxel and doxorubicin, two of the most effective drugs for ovarian cancer in current clinical use.

TABLE 9
Anticancer activity of YW-391 in mice bearing SKOV-3 human
ovarian cancer*

| | Dose | | % Tumor growth | % Maximum |
|---|---|---|---|---|
| Drug | (mg/kg) | Schedule | inhibition | weight loss |
| YW-391 | 0.13 | 15, 24, 33 | 52 | −3 |
| Paclitaxel | 25 | 15, 24, 33 | 22 | −1 |
| Doxorubicin | 5 | 15, 24, 33 | 39 | −4 |

*CD₁ nude female mice (7-9/group) were implanted s.c. with 5 × 10⁶ cells on day 0. Drugs were given i.v. on days 15, 24 and 33. For all tested drugs, P < 0.01 compared with non-drug

treated animals. YW-391 and doxorubicin were administered iv., and paclitaxel was administered ip.
Equivalents

From the foregoing detailed description of the specific embodiments of the invention, it should be apparent that unique antiproliferative compounds and unique synthetic procedures for producing the same have been described, resulting in therapeutic compounds effective against mammalian cellular proliferative diseases, e.g., tumors and cancers. Although particular embodiments have been disclosed herein in detail, this has been done by way of example for purposes of illustration only, and is not intended to be limiting with respect to the scope of the appended claims that follows. In particular, it is contemplated by the inventor that substitutions, alterations, and modifications may be made to the invention without departing from the spirit and scope of the invention as defined by the claims. For instance, the choice of CC-1065 derivative, or the use of particular linker molecules, or the choice of doses, or the choice of route of administration of the compositions of the present invention are believed to be matters of routine for a person of ordinary skill in the art with knowledge of the embodiments described herein.

1. A compound comprising a structure having the following formula:
CC-1065 analogue-linker-maleimide (Formula I)
or a pharmaceutically acceptable salt thereof, wherein:
A. the linker is selected from the group consisting of —C(O)R₁—, —C(O)OR₁—, —C(O)NR₂R₃—, —C(O)(CH₂)ₙ₁(OCH₂CH₂)ₙ₂—, where ₙ₁ is 1-6, and ₙ₂ is 0-20;
—C(O)(CH₂)ₙ₁R₄(OCH₂CH₂)ₙ₂— where ₙ₃ and ₙ₄ are independently 0-10;
—C(O)(CH₂)ₙ₁R₄(OCH₂CH₂)ₙ₂— where ₙ₁ is 1-6, and ₙ₂ is 0-20;
—C(O)(CH₂)ₙ₁(OCH₂CH₂)ₙ₂R₄— where ₙ₁ is 1-6, and ₙ₂ is 0-20;
—C(O)NR₂R₃(CH₂)ₙ₃R₄(CH₂)ₙ₄— where ₙ₃ and ₙ₄ are independently 0-10; and
—C(O)NR₂R₃(CH₂)ₙ₅(OCH₂CH₂)ₙ₆— where ₙ₅ and ₙ₆ are independently 0-10; and
wherein
R₁ is alkyl or aryl;
R₂ and R₃ are independently H, alkyl or aryl; but R₂ and R₃ cannot simultaneously be aryl;
R₄ is a valence bond, aryl, or alkyl containing at least one nitrogen;
B. the CC-1065 analogue comprises a compound with the following structure (Formula II),
wherein: 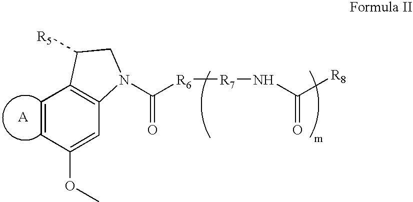
A is a 5-6 member ring alkyl, aryl or heteroaryl;
R₅ is CH₂Cl, CH₂Br, CH₂I or CH₂OSO₂CH₃;
R₆ is a valence bond, a C₁-C₆ alkyl, a C₂-C₆ alkenyl, a C₂-C₆ alkynyl or an aryl;
R₇ and R₈ are independently selected from aryl or heteroaryl; and
M is 0-2; and
C. maleimide.

2. The compound of claim 1, wherein the CC-1065 analogue comprises a compound having the structure of
formula IV, V or VI: 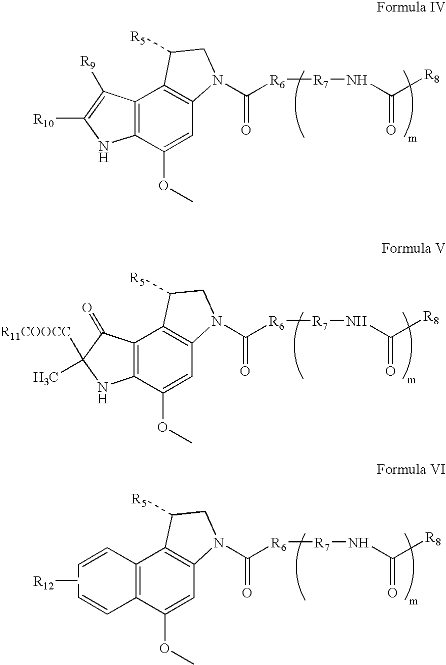
wherein:
R₅ is CH₂Cl, CH₂Br, CH₂I or CH₂OSO₂CH₃;
R₆ is a valence bond, a C₁-C₆ alkyl, a C₂-C₆ alkenyl, a C₂-C₆ alkynyl or an aryl;
R₇ and R₈ are independently selected from aryl or heteroaryl;
M is 0-2;
R₉ is H, C₂-C₆ alkyl, C(O)-alkyl, C(O)O-alkyl;
R₁₀ is H, C₂-C₆ alkyl;
R₁₁ is CH₃ or CF₃;
R₁₂ is H, NH₂, NO₂, O-alkyl, NH-alkyl, N(alkyl)₂, NHC(O)-alkyl, ONO₂, F, Cl, Br, I, OH, OCF₃, OSO₂CH₃, CO₂H, CO₂-alkyl, CO₂CF₃ or CN.

3. The compound of claim 2, wherein the CC-1065 analogue comprises a compound having the structure of
formula IV: 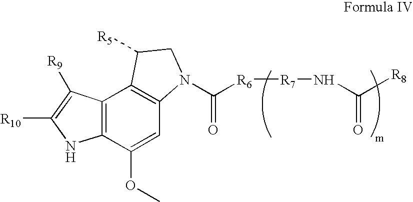
wherein:
R₅ is CH₂Cl, CH₂Br, CH₂I or CH₂OSO₂CH₃;
R₆ is a valence bond, a C₁-C₆ alkyl, a C₂-C₆ alkenyl, a C₂-C₆ alkynyl or an aryl;
R₇ and R₈ are independently aryl or heteroaryl;
M is 0-2;
R₉ is H, C₂-C₆ alkyl, C(O)-alkyl, C(O)O-alkyl.
R₁₀ is H, C₂-C₆ alkyl.

4. The compound of claim 2, wherein the CC-1065 analogue comprises a compound having the structure of
formula V: 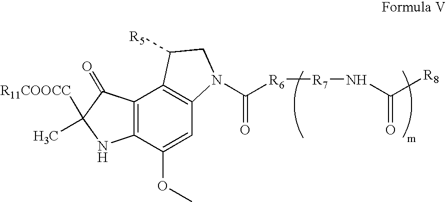
wherein:
R₅ is CH₂Cl, CH₂Br, CH₂I or CH₂OSO₂CH₃;
R₆ is a valence bond, a C₁-C₆ alkyl, a C₂-C₆ alkenyl, a C₂-C₆ alkynyl or an aryl;
R₇ and R₈ are independently aryl or heteroaryl;
M is 0-2;
R₁₁ is CH₃ or CF₃.

5. The compound of claim 2, wherein the CC-1065 analogue comprises a compound having the structure of
formula VI: 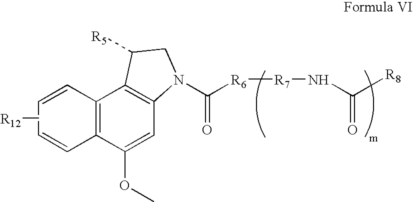
wherein:
R₅ is CH₂Cl, CH₂Br, CH₂I or CH₂OSO₂CH₃;
R₆ is a valence bond, a C₁-C₆ alkyl, a C₂-C₆ alkenyl, a C₂-C₆ alkynyl or an aryl;
R₇ and R₈ are independently aryl or heteroaryl;
R₁₂ is H, NH₂, NO₂, O-alkyl, NH-alkyl, N(alkyl)₂, NHC(O)-alkyl, ONO₂, F, Cl, Br, I, OH, OCF₃, OSO₂CH₃, CO₂H, CO₂-alkyl, CO₂CF₃ or CN.

6. The compound of claim 1, wherein the linker is —C(O)R₁— or —C(O)OR₁— and where R₁ is alkyl.

7. The compound of claim 1, wherein the linker is —C(O)NR₂R₃— where R₂ and R₃ are independently H or alkyl, but R₂ and R₃ are not H at the same time.

8. The compound of claim 1, wherein the linker is —C(O)(CH₂)ₙ₁(OCH₂CH₂)ₙ₂— where ₙ₁ is 1-6, and ₙ₂ is 0-20.

9. The compound of claim 1, wherein the linker is —C(O)(CH₂)ₙ₃R₄(CH₂)ₙ₄— and where ₙ₃ and ₙ₄ are independently 0-10 and R₄ is aryl or alkyl containing at least one nitrogen.

10. The compound of claim 1, wherein the linker is —C(O)(CH₂)ₙ₁R₄(OCH₂CH₂)ₙ₂— and where ₙ₁ is 1-6, and ₙ₂ is 0-20 and R₄ is a valence bond, aryl, or alkyl containing at least one nitrogen.

11. The compound of claim 1, wherein the linker is —C(O)(CH₂)ₙ₁(OCH₂CH₂)ₙ₂R₄— where ₙ₁ is 1-6, ₙ₂ is 0-20, and R₄ is a valence bond, aryl, and alkyl containing at least one nitrogen.

12. The compound of claim 1, wherein the linker is —C(O)NR₂R₃(CH₂)ₙ₃R₄(CH₂)ₙ₄— and
wherein
ₙ₃ and ₙ₄ are independently 0-10;
R₂ and R₃ are independently H or alkyl but R₂ and R₃ are not H at the same time;
R₄ is aryl, and alkyl containing at least one nitrogen.

13. The compound of claim 1, wherein the linker is —C(O)NR₂R₃(CH₂)ₙ₅(OCH₂CH₂)ₙ₆— and wherein
ₙ₅ and ₙ₆ are independently 0-10, and
R₂ and R₃ are independently H or alkyl but R₂ and R₃ are not H at the same time.

14. The compound of claim 5, wherein the linker is —C(O)R₁— and R₁ is alkyl; and wherein
R₅ is CH₂Cl, CH₂Br, CH₂I or CH₂OSO₂CH₃;
R₆ is a valence bond, a C₁-C₆ alkyl, a C₂-C₆ alkenyl, a C₂-C₆ alkynyl or an aryl;
R₇ and R₈ are independently aryl or heteroaryl;
R₁₂ is H, NH₂, NO₂, O-alkyl, NH-alkyl, N(alkyl)₂, NHC(O)-alkyl, ONO₂, F, Cl, Br, I, OH, OCF₃, OSO₂CH₃, CO₂H, CO₂-alkyl, CO₂CF₃ or CN.

15. The compound of claim 14, wherein R₆ is a valence bond or a C₂-C₆ alkenyl, where R₇ and R₈ are independently selected from aryl or heteroaryl, and where R₁₂ is H.

16. The compound of claim 15, wherein R₆ is a valence bond or CH═CH, and R₇ and R₈ are independently heteroaryl.

17. The compound of claim 16, wherein the CC-1065 analogue comprises a compound having the formula (+)-YW-391:

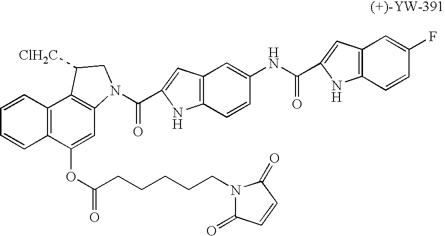

18. The compound of claim 16, wherein the wherein the CC-1065 analogue comprises a compound having the formula (+)-YW-392:

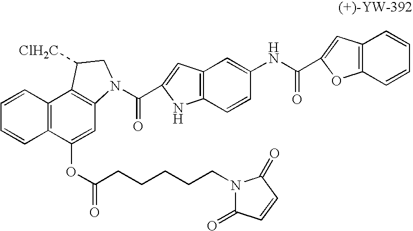

19. A method for treating cancer in a subject comprising administering to a subject having cancer, from 1 microgram/kg to 100 micrograms/kg of the compound of claim 1, wherein the proliferation of the cancer is inhibited.

20. A method for treating cancer in a subject comprising administering to a subject having cancer, from 1 microgram/kg to 100 micrograms/kg of the compound of claim 17, wherein the proliferation of the cancer is inhibited.

21. A method for treating cancer in a subject comprising administering to a subject having cancer, from 1 microgram/kg to 100 micrograms/kg of the compound of claim 18, wherein the proliferation of the cancer is inhibited.
